(19) United States
(12) Patent Application Publication
(10) Pub. No.: US 2022/0109157 A1
(43) Pub. Date: Apr. 7, 2022

(54) Conducting redox oligomers

(71) Applicants: Martin SJÖDIN, Knivsta (SE); Christian STRIETZEL, Uppsala (SE); Rikard EMANUELSSON, Uppsala (SE)

(72) Inventors: Martin SJÖDIN, Knivsta (SE); Christian STRIETZEL, Uppsala (SE); Rikard EMANUELSSON, Uppsala (SE)

(21) Appl. No.: 17/428,746

(22) PCT Filed: Feb. 7, 2020
(86) PCT No.: PCT/SE2020/050121

§ 371 Date: Aug. 5, 2021

(30) Foreign Application Priority Data
Feb. 7, 2019 (SE) 1950142-8

(51) Int. Cl. H01M 4/60; C07D 495/04; C07D 333/32; C08G 61/12; C09D 165/00; H01M 4/66

(52) U.S. Cl. CPC H01M 4/608; H01G 11/68; C07D 333/32; C08G 61/126; C09D 165/00; H01M 4/663; C08G 2261/124; C08G 2261/145; C08G 2261/143; C08G 2261/148; C08G 2261/1422; C08G 2261/1426; C08G 2261/1428; C08G 2261/18; C08G 2261/228; C08G 2261/226; C08G 2261/3243; C07D 495/04

(57) ABSTRACT

The present disclosure relates to compounds of formula IVa or IVb, or salts thereof, as intermediates in the manufacture of conducting redox polymers. L is a covalent linker moiety and R is a reversible redox group.

The disclosure further relates to conducting redox polymers produced from such compounds, as well as substrates coated with such conducting redox polymers, and organic batteries comprising such conducting redox polymers.

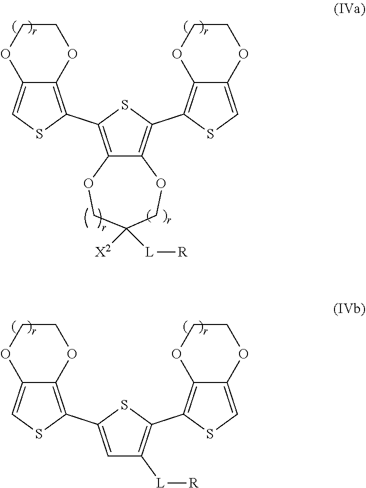

### TECHNICAL FIELD

The present invention relates to conducting redox oligomers for the manufacture of conducting redox polymers, and conducting redox polymers manufactured from such oligomers. The present invention further relates to coating compositions comprising conducting redox oligomers, and substrates coated with conducting redox polymers.

### BACKGROUND ART

There is an increasing demand for thin, flexible batteries, driven in part by a trend towards miniaturized, flexible and portable products and the need for thin and flexible energy storage systems for powering such products. For example, within the rapidly expanding smart packaging market segment there is a need for sensors, printed displays, circuits, anti-theft devices, RFID tags and smart labels, all of which will require powering by thin, flexible batteries. Flexible batteries typically utilize tradition cell chemistries such as lithium-ion or zinc carbon, and may be produced by using a polymer electrolyte arranged between flexible electrode substrates.

So-called conducting redox polymers have also been investigated with a view to applications in flexible electronics. These conducting redox polymers comprise a conducting polymer backbone and redox-active pendant groups. The conductive polymer makes the materials electrically conductive, which is a prerequisite for the material to function as battery material, while the redox-active pendant groups allow the material to store charge. Although a major application for this type of polymers is in batteries, conducting polymers with redox moieties could also be applicable in other fields, such as catalysis and fuel cells.

There remains a need for improved materials for flexible energy storage applications and improved means of manufacturing such materials.

### SUMMARY OF THE INVENTION

The inventors of the present invention have identified a number of shortcomings in prior art flexible energy sources and materials for manufacturing such.

Flexible batteries manufactured using traditional battery chemistries such as lithium-ion or zinc carbon chemistries are non-combustible and comprise significant quantities of metals, meaning that such batteries are not readily disposable. Instead, such batteries require recycling or careful deposition. This limits the market for such batteries as recycling or deposition of the batteries is not always economically viable or technically feasible.

Batteries composed of conducting redox polymers do not suffer from these problems and may be disposed as normal household waste, for example by incineration, thus significantly expanding the potential market of such batteries. However, known conducting redox polymers are difficult to manufacture and suffer from poor processability, thus limiting their utility. Methods of improving the processability of the polymers result in polymers having poor atom economy, lower capacity per unit mass, and thus worsened economic viability.

It is an object of the present invention to provide means for manufacturing flexible energy storage devices that overcome or at least alleviate one or more of the above mentioned drawbacks. In particular, it is an object of the present invention to provide a means of facilitating the manufacture and processability of conducting redox polymers.

These objects are achieved by a compound, or a salt thereof, according to the appended independent claims.

The compound has a formula IVa or IVb:

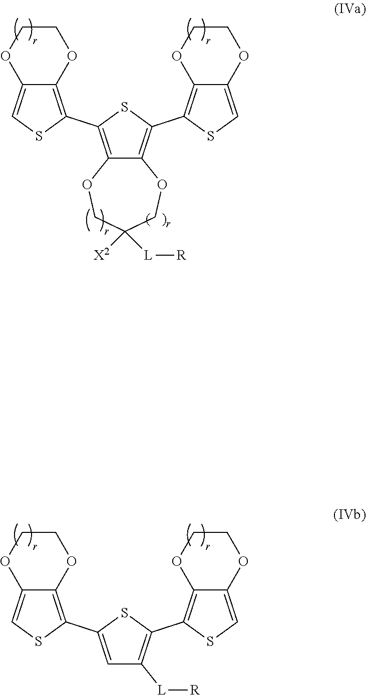

Each instance of -L- is independently selected from a direct bond or a covalent linker moiety.

Each instance of —R is independently a reversible redox group.

Each instance of —X² is independently selected from -L-H, -L-T, or -L(-R)ₘ.

Each instance of T is independently selected from —CN or —N₃.

Each instance of m is independently selected from 1 to 5.

Each instance of r is independently selected from 0, 1 or 2.

Such compounds are henceforth termed conducting redox oligomers. The conducting redox oligomers are readily soluble in a range of solvents, and are therefore simple to process. For example, the conducting redox oligomers may easily be coated or printed on a substrate. The conducting redox oligomers are furthermore easily polymerized to conducting redox polymers, either by mild chemical oxidation or oxidative electropolymerization at relatively low potential. They may be polymerized in the solid state, meaning that the material may first be processed as an oligomer and then polymerized once no further processing is required. Thus, the present invention bypasses the problem of poor processability of the conducting redox polymers. On the other hand, monomers corresponding to the conducting redox oligomers (i.e. compounds of formula I having n=1) require harsh oxidizing conditions in order to achieve polymerization and they can, in general, not be polymerized in solid state. The polymers resulting from such polymerization are largely insoluble and not readily processable.

According to a further aspect of the invention, the objects of the invention are achieved by a polymer according to the appended independent claims.

The polymer comprises a repeating unit of formula RIVa, or a salt thereof:

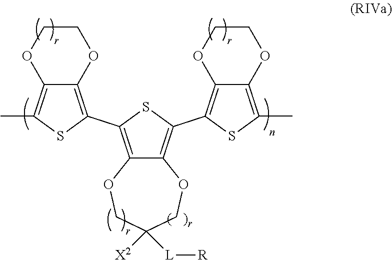

n is from 2 to 5, such as 3 or 5, preferably 3.

Each instance of —X² is independently selected from -L-H, -L-T, or -L(-R)ₘ.

Each instance of -L- is independently selected from a direct bond or a covalent linker moiety.

Each instance of -T is independently selected from —CN or —N₃.

Each instance of —R is independently a reversible redox group.

Each instance of r is independently selected from 0, 1 or 2.

Each instance of m is independently selected from 1 to 5.

According to yet another aspect of the invention, the objects of the invention are achieved by a polymer obtained by oxidative polymerisation of one or more compounds as described herein.

The following features are readily applicable to all compounds and polymers as described herein, where appropriate. This includes, but is not limited to, compounds of formula IVa or IVb, as well as polymers obtained by oxidative polymerisation of such compounds, or polymers comprising a repeating unit RIVa.

Each instance of —X² may independently be selected from —H, C₁-C₁₂ alkyl, or -L(-R)ₘ. For example, each instance of X₂ may be independently selected from H or C₁-C₁₂ alkyl.

The compound may have a formula IVa:

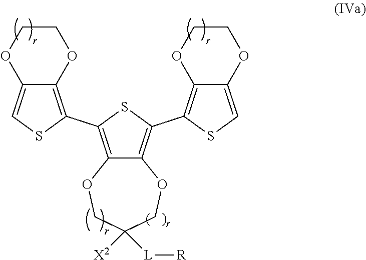

wherein each instance of r is independently selected from 0, 1 or 2.

Each instance of -L- may independently be selected from a covalent linker moiety having a structure —(CH₂)ₛ-G¹-(CH₂)ₜ-G²- or -G²-(CH₂)ₜ-G¹-(CH₂)ₛ—, wherein s is from 0 to 6, t is from 0 to 6, and each of -G¹- and -G²- is independently selected from the group consisting of a direct bond, —O—, —S—, —SO₂—, —SO₃—, —O₃S—, —SO₂NH—, —NHSO₂—, —NH—, —N(C₁-C₆ alkyl)- , —C(O)—, —CO₂—, —O₂C—, —C(L)NH—, —NHC(O)—, —OC(O)O—, —NHC(O)NH—, —NHC(O)O—, —OC(O)NH——C≡C—, —CH═CH—, —Ph— and —Hy—.

Each instance of R may independently be an organic redox group.

Each instance of R may independently be selected from the group consisting of terephthalate, naphthoquinone, anthraquinone, catechol, quinone, quinizarin, naphthazarin, indigo, TEMPO (2,2,6,6-tetramethylpiperidine-1-oxyl), galvinoxyl, phenol, naphthalene diimide, pyrene diimide, perylene dimide, dibenzothiophenesulfone , or substituted derivatives thereof.

Alternatively, each instance of R may be an organometallic redox catalyst.

According to yet a further aspect of the invention, the objects of the invention are achieved by a method of manufacturing a polymer-coated substrate according to the appended independent claims.

The method comprises the following steps:

a) providing a substrate;

b) coating a compound according to any one of claims 1-9 onto the substrate in order to produce a substrate having an oligomeric coating; and

c) polymerising the oligomeric coating by oxidative polymerization to provide a polymer-coated substrate.

Instead of trying to process the conducting redox polymer which has limited solubility and is difficult to work with, this method allows the substrate to be instead coated with a conducting redox oligomer as described herein. This significantly facilitates the processablility of the coating layer of the substrate. Once the coating layer has been provided, the conducting redox oligomer is readily polymerisable in the solid state in order to provide a substrate coated with the conducting redox polymer.

The polymerisation may be performed by electropolymerisation techniques, or by chemical oxidative polymerisation techniques.

According to yet a further aspect of the invention, the objects of the invention are achieved by a start material for a polymerization reaction, the start material comprising a redox oligomer as described herein. The polymerisation reaction may be a chemical oxidative polymerisation reaction or an oxidative electropolymerisation reaction.

According to yet another aspect of the invention, the objects of the invention are achieved by a coating composition comprising a compound as described herein.

According to yet a further aspect of the invention, the objects of the invention are achieved by a polymer-coated substrate comprising a polymer as described herein. The substrate may be a conducting current collector material, such as graphite. The substrate may be a porous substrate, such as a porous conducting substrate, such as a porous graphite substrate.

According to yet a further aspect of the invention, the objects of the invention are achieved by an organic battery comprising a compound as described herein, and/or a polymer as described herein, and/or a polymer-coated substrate as described herein.

The organic battery may have an aqueous electrolyte, such as an aqueous acid electrolyte.

Further objects, advantages and novel features of the present invention will become apparent to one skilled in the art from the following detailed description.

### BRIEF DESCRIPTION OF THE DRAWINGS

For a fuller understanding of the present invention and further objects and advantages of it, the detailed description set out below should be read together with the accompanying drawings, in which the same reference notations denote similar items in the various diagrams, and in which:

FIG. 1 illustrates a range of conducting oligomer backbones that comprise a functional handle for attachment of pendant groups;

FIG. 2 illustrates synthetic methods for manufacturing oligomer backbones comprising a functional handle;

FIG. 3 illustrates synthetic methods for manufacturing conducting redox oligomers by attachment of a redox group to an oligomer backbone comprising a functional handle;

FIG. 4 illustrates a synthetic method for manufacturing conducting redox oligomers from a monomer already comprising a redox group;

FIG. 5 illustrates a range of conducting redox oligomers obtainable by the methods illustrated in FIGS. 2-4;

FIG. 6a is a cyclic voltammogram of poly-E-PMeSHQ-E in 0.5 M sulphuric acid;

FIG. 6b is a graph showing the results of measurements using an interdigitated array electrode to measure electric conductivity of poly-E-PMeSHQ-E as a function of potential (redox state) and temperature;

FIG. 7 is a is a cyclic voltammogram illustrating the progressive electropolymerisation of E-PMeSHQ-E;

FIG. 8a is a cyclic voltammogram of poly- E-PMeO(C═O)NQ-E in 0.5 M sulphuric acid;

FIG. 8b is a graph showing the results of measurements using an interdigitated array electrode to measure electric conductivity of poly- E-PMeO(C═O)NQ-E as a function of potential (redox state) and temperature;

FIG. 9a is a cyclic voltammogram comparing the oxidation potential of monomeric EMeCCHQ to oligomeric E-PMeSHQ-E;

FIG. 9b is a diagram illustrating the oxidation potentials of a range of thiophene monomers and trimers; and

FIG. 10 is a cyclic voltammogram of porous conducting redox polymer composites.

### DETAILED DESCRIPTION

The conducting redox oligomers of the invention facilitate the manufacture of components for energy storage comprising conducting redox polymers. They do so by allowing the material to be processed as the conducting redox oligomer prior to facile solid-state polymerisation to the conducting redox polymer. In this manner, the processability problems associated with conducting redox polymers are avoided to a significant extent.

The inventors have identified the following aspects as meriting consideration in seeking to achieve a functional conducting redox polymer suitable for use as battery active material.

Proccessability: In order to be able to achieve thicker electrode layers without binder, the precursor for the polymer should be easily be coated on the surface and subsequently polymerized. It is preferable that the polymerization may be performed in aqueous media, thus reducing the need for organic solvents and simplifying the production process.

Potential matching: Since all conducting properties are given by the conducting polymer backbone, the pendent group should have redox properties in the potential region where the conducting polymer actually is conducting. This is termed potential matching.

Well-defined voltage output from pendent: The pendent group that is attached to the conducting polymer should provide provide a well-defined voltage output, so it can be used as an efficient electrode in a battery.

Compatible with an aqueous electrolyte: Since the conducting redox polymer is intended for use as electrode active material in an aqueous organic battery the pendent group should be i) within the potential window of water to eliminate side reactions and ii) sufficiently hydrophilic so that ion transport can occur.

### Thiophene Oligomer Backbone

The backbone of the conducting redox oligomers is based on oligomers of substituted or unsubstituted thiophenes. By substituted or unsubstituted thiophene it is meant any thiophene moiety that may be utilized as a backbone in a conducting polymer. Such thiophenes include but are not limited to thiophene and dioxythiophenes such as MDOT (methylene 3,4-dioxythiophene), EDOT (ethylene 3,4-dioxythiophene), ProDOT (propylene 3,4-dioxythiophene), BueDOT (butylene 3,4-dioxythiophene) and PheDOT (phenylene 3,4-dioxythiophene). Note that the term “unsubstituted thiophene” denotes that the oligomer backbone is based on thiophene per se, not a substituted thiophene derivative such as a dioxythiophene. However, the “unsubstituted thophene” oligomer still comprises pendant groups as indicated by the formulas herein.

The outer thiophene moieties of the conducting redox oligomer are selected from MDOT, EDOT and/or ProDOT. The central thiophene moiety is thiophene or an alkylene dioxythiophene moiety, such as ProDOT. The pendant redox group is directly or indirectly covalently bound to the central thiophene moiety.

The oligomer backbone is a trimer. Ease of oxidation to some extent corresponds to the length of oligomer and a monomer or dimer would thus requires harsher polymerization conditions than the corresponding trimer.

All thiophenes in the oligomer backbone may be the same or different. For example, the thiophenes may alternate between a first and a second thiophene (e.g. a trimer comprising a M²-M¹-M² backbone where M¹ and M² are different thiophenes). In such a case, the central thiophene may preferably be EDOT or ProDOT for ease of synthesis of the conducting redox oligomer.

### Pendant Redox Group

At least one pendant redox group —R is directly or indirectly covalently bonded to the oligomeric backbone. The pendant redox group may be any redox group being reversibly redox active. By reversible redox group, it is meant that the “free” redox group (i.e. redox molecule in solution, not bound to any polymer or oligomer) is not consumed or destroyed in the reduction/oxidation step and that this step may be reversed and repeated for a plurality of redox cycles (i.e. two or more redox cycles). For example, a group should be able to withstand each redox cycle (reduction followed by oxidation or alternatively oxidation followed by reduction) with a yield of at least 80% or more, such as at least 90% or more, at least 95% or more, or at least 99% or more.

The reversible redox group may be reduced/oxidized within a potential interval of from −3.25 to 1.5 V verses NHE (normal hydrogen electrode) in aqueous solution at standard temperature and pressure (STP 0° C., 1 Bar). The potential vs. NHE is for this purpose considered to be equivalent to the potential vs. SHE (standard hydrogen electrode). For example, when measured at STP in 0.5M aqueous H₂SO₄ a reduction/oxidation potential of from −1.5 V to 1.5 V vs. NHE. When measured at standard temperature and pressure (0° C., 1 Bar) in 1M aqueous NaCl a reduction/oxidation potential of from −1.5 V to 1.5 V vs. NHE. When measured in organic solution, such as propylene carbonate (PC), acetonitrile, or 1M LiPF₆ at STP, a reduction/oxidation potential of from 0 V to 4.75 V vs. Li. In this context it is the redox potential of the “free” redox group that is meant, not the redox group bound to an oligomer or polymer. Electrochemical techniques for measuring redox potential are known in the art, and lists of standard reduction potentials (E^{r}₀) and oxidation potentials (E^{o}₀) are readily available. Note that the potential may be measured in relation to another reference electrode such as the saturated calomel electrode (SCE) and related to NHE by the known correlation between the reference electrodes. For example, SCE has a potential of +0.241 V in relation to SHE and an Ag/AgCl reference electrode has a potential of +0.197 in relation to SHE.

The redox group may preferably be an organic redox group or a redox catalyst, such as an organometallic redox catalyst. Organic redox groups have the advantage of being metal-free and thus are relatively environmentally benign and readily disposable in household waste. Redox catalysts, such as organometallic redox catalysts, provide chemically addressable sites on the polymer backbone and give rise to potential applications such as within sensor and fuel-cell technologies.

Preferably, the organic redox groups are selected from terephthalate, naphthoquinone, anthraquinone, catechol, quinone, quinizarin, naphthazarin, indigo, TEMPO, galvinoxyl, phenol, naphthalene diimide, pyrene diimide, perylene dimide, and dibenzothiophenesulfone groups, or substituted derivatives thereof. By substituted derivatives thereof, it is meant derivatives comprising one or more appropriate organic substituent groups, such as from one to five independently selected organic substituent groups. Appropriate organic substituent groups include but are not limited to —F, —Cl, —Br, —I, —C₁-C₆ alkyl, —OH, —SH, —O(C₁-C₆ alkyl), —S(C₁-C₆ alkyl), —SO₂H, —SO₃H, —SO₂NH₂, —NH₂, —NH(C₁-C₆ alkyl), —N(C₁-C₆ alkyl)₂, —C(O)H, —CO₂H, —CO₂(C₁-C₆ alkyl) , —C(O)NH₂, —NHC(O)H, —NHC(O)—C₁-C₆ alkyl, —NHC(O)NH₂, —OC(O)NH——C≡CH, —CH═CH₂, —Ph and —Hy. Substituents such as electron-donating, electron-withdrawing or sterically hindering substituents may be used on the organic redox groups to tune the redox properties of the group, as known in the art.

The pendant redox group is covalently linked to the oligomer backbone by a linker moiety -L-. Each individual linker moiety may link more than one pendant redox group to the oligomer backbone, up to a limit of five redox groups per linker. The linker moiety may be a direct bond, i.e. the redox group may be directly bonded to the oligomer backbone, or it may be a straight, branched or cyclic covalent moiety comprising atoms selected from C, H, N, O and S and linking the oligomer backbone to one or more redox groups. For example, the linker moiety may have a structure —(CH₂)ₛ-G¹—(CH₂)ₜ-G²- or -G²-(CH₂)ₜ-G¹-(CH₂)ₛ—. In such a case, s may be from 0 to 6, t may be from 0 to 6, and each of -G¹- and -G²- may independently be selected from the group consisting of a direct bond, —O—, —S—, —SO₂—, —SO₃—, —O₃S—, —SO₂NH—, —NHSO₂—, —NH—, —N(C₁-C₆ alkyl)-, —C(O)—, —CO₂—, —O₂C—, —C(O)NH—, —NHC(O)—, —OC(O)O—, —NHC(O)NH—, —NHC(O)O—, —OC(O)NH——C≡C—, —CH═CH—, —Ph— and —Hy—.

The abbreviation “Hy” herein denotes a heterocyclic moiety. As used herein, the term “heterocyclic” refers to organic compounds containing at least one atom of carbon, and at least one element other than carbon, such as sulfur, oxygen or nitrogen within a ring structure. These structures may comprise either simple aromatic rings or non-aromatic rings. The ring structure may be mono-cyclic or bi-cyclic or polycyclic. Each mono-cyclic ring may be aromatic, saturated or partially unsaturated. A bi-cyclic ring system may include a mono-cyclic ring containing one or more heteroatoms fused with a further mono-cyclic heterocycle, cycloalkyl or carboaryl group.

The conducting redox oligomer comprises at least one pendant redox group, but may comprise a multitude of pendant redox groups. The redox groups in the conducting redox oligomer may be the same or they may be different. Each substituted or unsubstituted thiophene in the oligomer backbone may comprise up to two linker moieties, and each linker moiety may comprise up to five redox groups. Thus, the maximum number of redox groups that the oligomer may comprise is 10n, wherein n is the number of thiophene units in the oligomer backbone. However, it is preferable that the oligomer comprises no more than a single redox group per linker, i.e. 2n redox groups, or no more than a single redox group per thiophene, i.e. 1n redox groups, or most preferably no more than a single redox group per oligomer.

A single redox group R may be used to connect two or more oligomers, each oligomer being connected to the redox group via a linker moiety -L- as described above. Oligomers connected in this manner may be incorporated into conducting redox polymers in order to provide cross-links between the formed polythiophene chains. Thus, by using a desired ratio of non-crosslinked to crosslinked oligomer, the mechanical properties of the formed polymer may be tailored.

### Further Groups

The oligomer backbone may be further substituted with one or more further groups. These groups may be used to tailor the properties of the oligomer or resulting polymer, or they may simply be present in order to provide a convenient synthetic route to the conducting redox oligomer. These further groups may for example comprise a linker moiety as described above, terminated by a hydrogen atom —H— or a terminal group -T. Suitable terminal groups include but are not limited to nitrile or azide groups. The further groups may for example be C₁-C₁₂ alkyl groups.

### Processing of the Conductive Redox Oligomer

The conductive redox oligomers as described above are soluble in a variety of solvents, including but not limited to polar aprotic solvents such as acetonitrile, NMP, DMSO and DMF. The resultant solutions may either be polymerised directly to provide a conducting redox polymer suspension suitable for further use, or they may first be processed prior to a final polymerization step. For example, a dispersion of the conductive redox polymer may be processed by coating or printing to a substrate. The substrate may for example be a conducting current collector material such as graphite. After depositing the conducting redox oligomer, it may be polymerized in the solid state. This may be done either by treatment with a mild chemical oxidant such as N-oxoammonium salts, or it may be performed by electropolymerisation using the substrate as an electrode. The oxidation potential of the conducting redox oligomer is lower than that of the corresponding monomer, meaning that relatively mild chemical oxidants (e.g. N-oxoammonium salts) or low oxidation potentials may be used. The conducting redox oligomers according to the present invention have suitably low oxidation potentials such that they may be polymerised in aqueous media. This permits deposition of the oligomer onto a substrate as an organic solution, followed by drying to provide a coating of the conducting redox oligomer. The conducting redox oligomer coating may subsequently be electropolymerised in aqueous media without dissolution or delamination of the coating layer. This greatly facilitates production of organic electronic devices, such as batteries, using the conducting redox oligomers.

### Conductive Redox Polymer

The conductive redox polymers obtained by polymerisation of the conductive redox oligomers display, as expected, both good conductivity and a possibility of storing charge by redox-reaction of the redox-active groups. The exact structure of the polymers obtained have not been determined. However, by analogy to polymerisation of the corresponding monomers, it can be assumed that a linear polythiophene is obtained. An exception is when crosslinking oligomers as described above are provided in the oligomer mixture to be polymerised. Here it can be assumed that the liner polythiophene chains are crosslinked to the relevant degree by bridging redox groups.

Note that when the thiophenes in the backbone differ from each other (e.g. a trimer comprising a M²-M¹-M² backbone where M¹ and M² are different thiophenes), or when the pendant groups attached to the thiophenes differ from each other (e.g. the oligomer comprises a single -L-R group arranged on the central thiophene of an odd-numbered oligomer), then a periodic copolymer is obtained, i.e. the polymer has units arranged in a repeating sequence. It is highly unlikely that such periodic polymers could be produced by traditional polymerisation techniques using the corresponding monomer units.

The polymer comprises at least two repeating units of formula RI or RIII, preferably at least 5 repeating units, more preferably at least 10 repeating units. Since the repeating unit is itself a thiophene n-mer, the number of thiophene units in the polymer is given by n* number of repeating units.

### Applications

The conducting redox polymers or substrates coated with such may find utility in a wide variety of applications. The polymers may be used in energy storage applications such as batteries or pseudocapacitors. Further potential uses include application as within sensors, electrocatalytic reactors (e.g. fuel cells), solar cells and transistors. The polymers have the advantage of being cheap, easily disposable and flexible, meaning that a great variety of potential applications exist.

The conducting redox polymers display conductance at a relatively low onset potential. This, together with the use of a pendant redox group matching the conductance properties of the polymer, permits the use of aqueous electrolytes in organic batteries. The use of aqueous electrolytes decreases the cost of manufacture and improves the ease of disposal of such batteries.

### Examples

The invention will now be described in more detail with reference to certain exemplifying embodiments and the drawings. However, the invention is not limited to the exemplifying embodiments discussed herein and/or shown in the drawings, but may be varied within the scope of the appended claims.

### General Synthesis of the Conducting Redox Oligomers

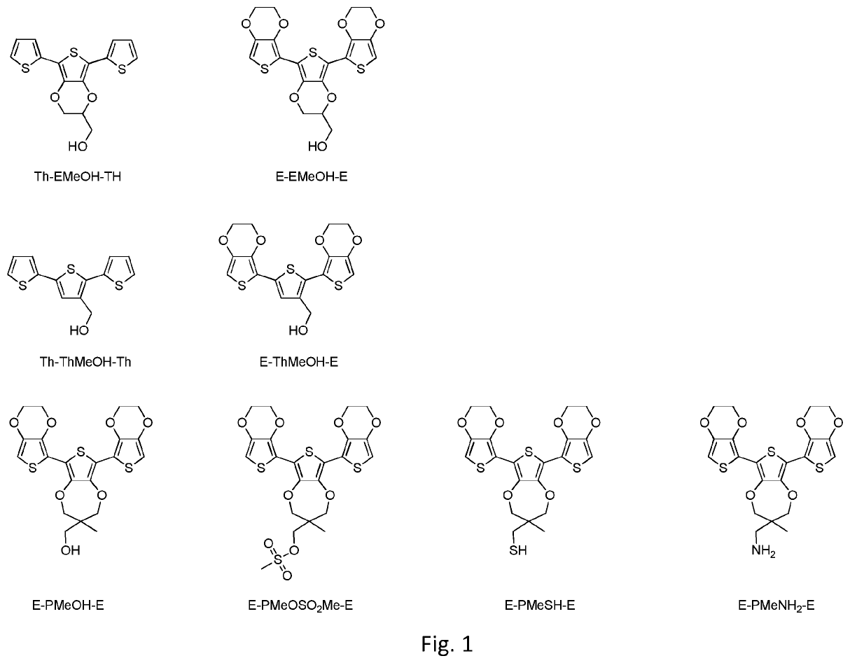

A variety of oligomeric backbones were synthesised, as illustrated in FIG. 1. A name for each backbone using the naming convention used herein is also provided. Each backbone is furnished with a suitable handle for further attachment of one or more redox groups.

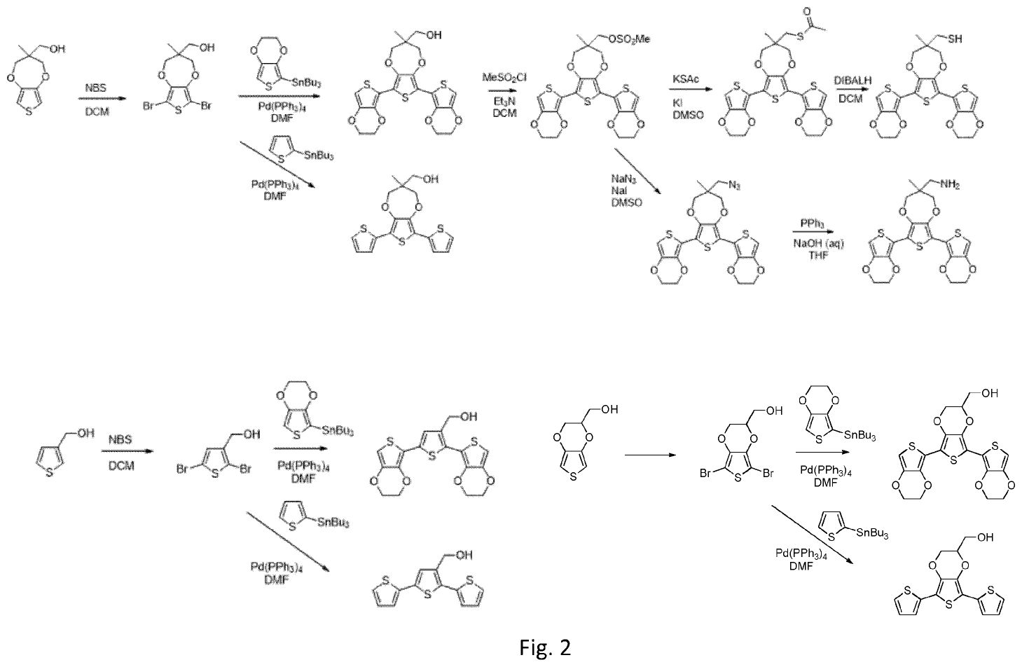

The oligomeric backbones as illustrated in FIG. 1 were synthesised using standard organic synthesis methods known in the art, as illustrated in FIGS. 2a for a variety of backbones and handles. The oligomers with a hydroxyl group handle were assembled from individual thiophenes by Stille coupling. The handle could then converted to a thiol by mesylation, nucleophilic substitution by thioacetate and reduction of the thioacetate to a thiol. Alternatively, the handle could be converted to an amine by mesylation, nuceophilic substitution by azide and Staudinger reaction to provide the amine.

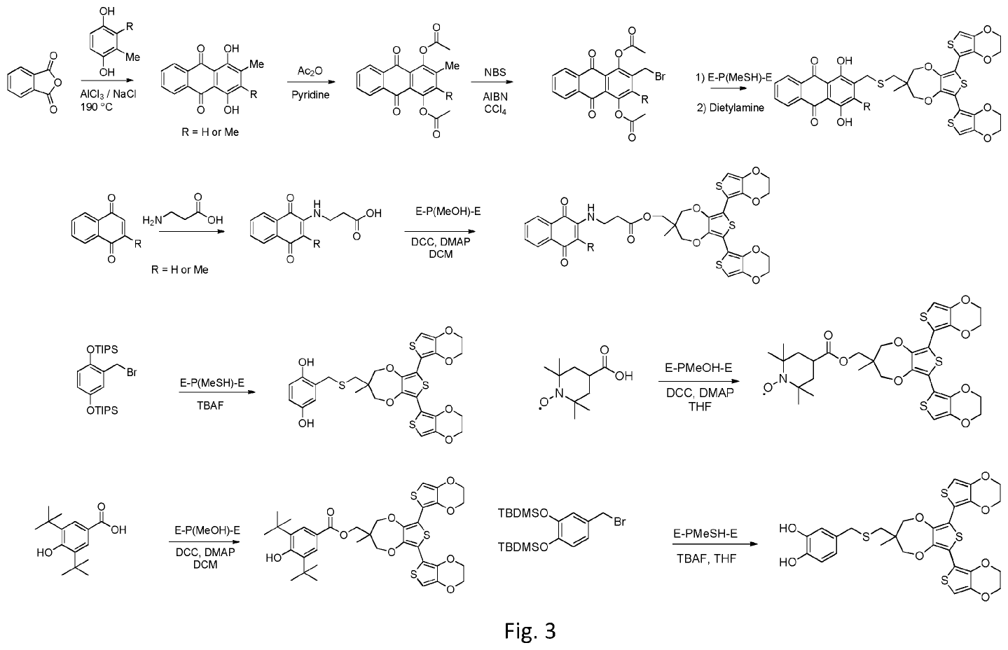

FIG. 3 illustrates the further elaboration of the oligomeric backbones to couple various redox pendant groups to the backbone via the handle already present. These couplings were performed using standard methods as known in the art.

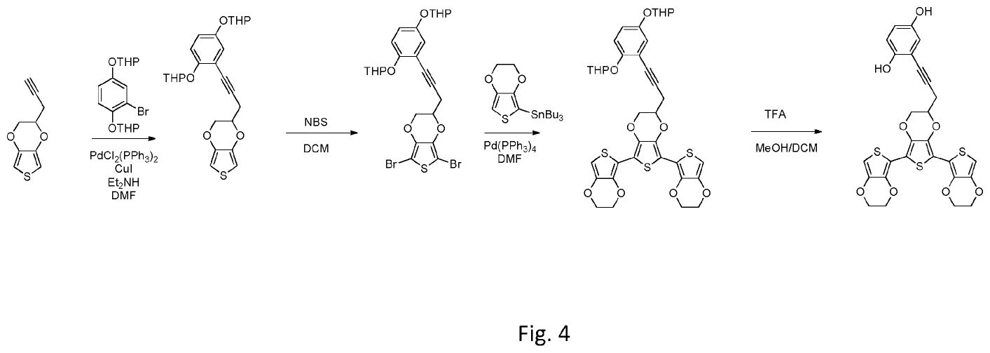

FIG. 4 illustrates an alternative means of synthesising the conducting redox oligomer by first coupling the redox group to a thiophene monomer by Sonogashira coupling, then assembling the oligomer units by Stille coupling.

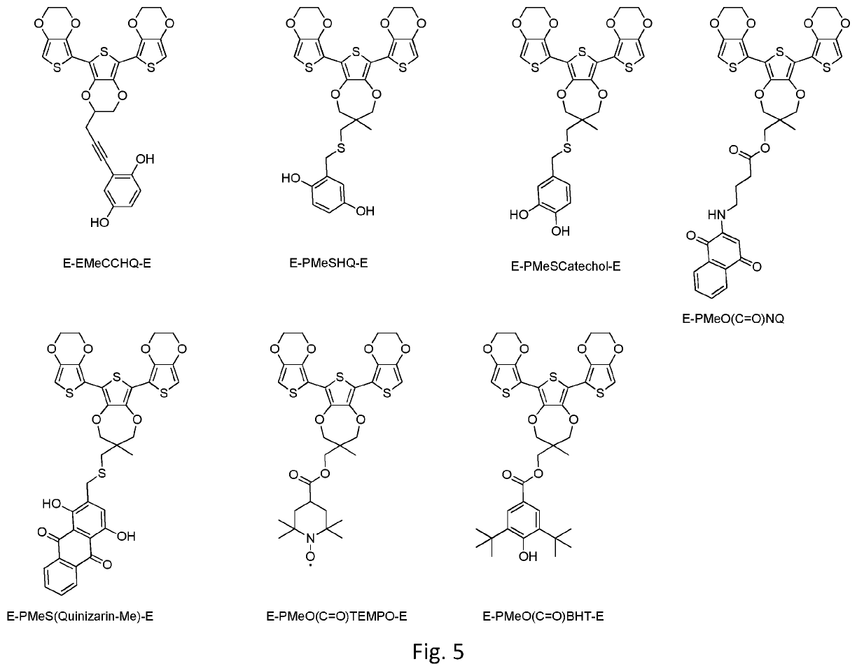

FIG. 5 illustrates a range of conducting redox oligomers synthesised by the methods described, as well as the naming conventions used for some of these conducting redox oligomers.

### General Procedure for Hydroxyl Functionalized Thiophene Trimers

As examples E-PMeOH-E, Th-EMeOH-E, E-ThMeOH-E, Th-PMeOH-Th and Th-ThMeOH-Th was assembled through the method below. In short, dibromination of the 2,5-positions of the hydroxy functionalized thiophene segment and subsequent Stille coupling of the dibromo species with 2-(tributylstannyl)thiophene, 2-(tributylstannyl)ethylenedioxythiophene or 2-(tributylstannyl)propylenedioxythiophene yield the hydroxy functionalized trimers.

The central segment (10 mmol, 1 eq) was dissolved in degassed DCM (100 mL) and NBS (21 mmol, 2.1 eq) was added. The reaction was stirred until consumption of starting materials (1-2 h for derivatives of EDOT or ProDOT, 14-18 h for thiophenes). The reaction mixture was directly added to silica, deactivated through addition of Et₃N, and purified through column chromatography (Pentane: EtOAc gradient from 0% EtOAc to 20%). The initially white solid darkened upon prolonged storage to give a black/blueish solid. Yield (80-90%). The solid was used immediately in the next step.

The dibromo central unit (10 mmol, 1 eq) and stannyl flanking unit (22 mmol, 2.2 eq) was placed in round bottle flask containing anhydrous DMF (75 mL) and degassed by bubbling Ar through the solution for 20 min. Then, Pd(PPh₃)₄ (0.75 mmol, 7.5 mol %) was added and the reaction flask placed in a pre-heated metal block (120° C.). The solution was stirred under Ar for 16 h. The DMF was removed under reduced pressure and the residue dissolved in EtOAc (150 mL) and filtered through celite. The organics was extracted with 1M HCl (aq), brine and the organics dried over MgSO4. Silica was added and the solvent removed under vacuum. The residue was purified through column chromatography (Pentane:EtOAc, Gradient from 5% EtOAc to 80%). Fractions containing product was evaporated, re-dissolved with minimal amount of DCM (10 mL) and dropwise added into a stirred solution of pentane (300 mL). The precipitate was filtered and dried under vacuum.

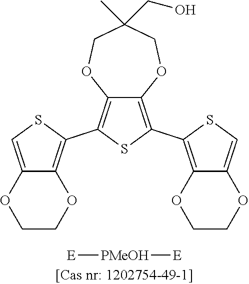

Yellow solid. Yield: 64%.

¹H NMR (500 MHz, CDCl₃) δ 6.26; (2H, s), 4.35; (4H, s), 4.23; (4H, m), 4.21; (2H, d, J=11.9) 3.87; (2H, s), 3.77; (2H, d, J=11.9), 0.96; (3H, s)

¹³C NMR (126 MHz, CDCl₃) δ 145.2, 141.0, 137.2, 112.8, 109.7, 97.6, 76.9, 65.5, 64.2, 63.5, 43.5, 15.6.

Consistent with Macromolecules 2010, 43, 1, 37-43.

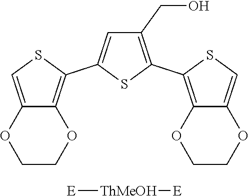

[2,5-bis(2,3-dihydrothieno[3,4-b][1,4]dioxin-5-yl)thiophen-3-yl]methanol

Brown solid. Yield: 60%.

¹H NMR (500 MHz, CDCl₃) δ 7.28; (1H, s), 6.39; (1H, s), 6.23; (1H, s), 4.61; (2H, s), 4.33; (2H, m), 4.28; (2H, m), 4.25; (4H, m).

¹³C NMR (126 MHz, CDCl₃) δ 142.0, 141.7, 139.2, 138.2, 138.0, 135.0, 128.3, 125.0, 112.0, 109.9, 100.0, 97.4, 65.2, 65.1, 64.7, 64.6, 59.5.

HRMS (ES+TOF) calcd for [C₁₇ H₁₄ O₅S₃+H, M+H]⁺: 395.0082; found: 395.0090.

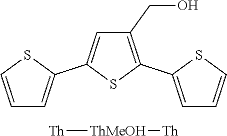

3′-(hydroxymethyl)-2,2T:5′,2″-terthiophene

Brown solid. Yield: 62%.

¹H NMR (500 MHz, CDCl₃) δ 7.23; (4H, m), 7.03; (2H, m), 4.45; (2H, m), 4.27; (1H, dd, J=12.0, 8.2 Hz), 3.97; (2H, m).

¹³C NMR (126 MHz, CDCl₃) 138.2, 136.8, 135.9, 134.7, 132.0, 128.0, 127.9, 126.4, 126.1, 125.7, 124.7, 123.7, 59.0

Consistent with Tet Lett, 2001, 49, 8733

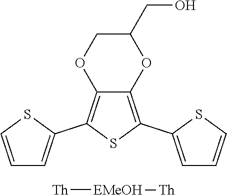

(5,7-di(thiophen-2-yl)-2,3-dihydrothieno[3,4-b][1,4]dioxin-2-yl)methanol

Brown solid. Yield: 55%.

¹H NMR (500 MHz, CDCl₃) δ 7.34; (1H, dd, J=5.1, 1.2 Hz), 7.24; (2H, m), 7.21; (1H, dd, J=3.6, 1.2 Hz), 7.18; (1H, dd, J=3.6, 1.1 Hz), 7.09; (1H, dd, J=5.1, 3.6 Hz), 7.02; (1H, dd, J=5.1, 3.6 Hz), 4.76; (2H, s).

¹³C NMR (126 MHz, CDCl₃) δ 137.1, 137.1, 134.4, 134.4, 128.0, 127.4, 127.3, 124.2, 124.1, 123.2, 123.0, 109.8, 109.8, 74.7, 66.1, 61.7.

Consistent with Org. Biomol. Chem. 2015,13, 8505-8511

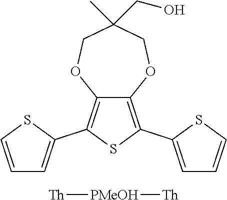

Beige solid. Yield: 65%

¹H NMR (500 MHz, CDCl₃) δ 7.24; (2H, d, J=5.0 Hz), 7.22; (2H, d, J=3.7 Hz), 7.02; (2H, dd, J=5.0, 3.7 Hz), 4.27; (2H d, J=12.1 Hz, 2H), 3.87; (d, J =12.1 Hz, 2H), 3.85; (2H, s), 1.03; (3H, s).

¹³C NMR (126 MHz, CDCl₃) δ 145.3, 134.6, 127.0, 124.7, 123.1, 114.7, 76.7, 65.7, 44.2, 17.1.

HRMS (ES+TOF) calcd for [C₁₇ H₁₆O₃ S₃+H, M+H]⁺: 365.0340; found: 365.0354.

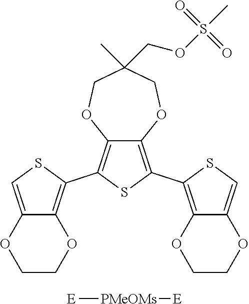

E-PMeOH-E (4.05 g, 8.4 mmol, 1 eq) was dissolved in DCM (100 mL) and triethylamine (1.29 mL, 9.3 mmol, 1.1 eq) was added. The solution was cooled using an ice/water bath and methanesulfonyl chloride (0.72 mL, 9.3 mmol, 1.1 eq) was added dropwise. After 20 min the ice/water bath was removed and the reaction was allowed to reach room temperature over 2 h. The reaction was quenched by addition of NaHCO₃ (sat). The organics were separated and washed with brine, dried over MgSO4, filtered and added to silica. Solvent was removed under vacuum and product was purified through column chromatography (Pentane:DCM, Gradient from 5% DCM to 80%). Fractions containing product was evaporated, re-dissolved in minimal amount of DCM (40 mL) and slowly added into a stirred solution of pentane (200 mL). The pale-yellow precipitate was filtered and dried under vacuum. Yield: 90%.

¹H NMR (500 MHz, CDCl₃) δ 6.28; (2H, s), 4.52; (2H, s), 4.36; (4H, m), 4.24; (4H, m), 4.22; (2H, d, J=12.1 Hz), 3.70; (2H, d, J=12.1 Hz), 3.10; (3H, s), 1.01; (3H, s).

¹³C NMR (126 MHz, CDCl₃) δ 144.2, 141.4, 137.6, 114.2, 110.1, 98.4, 76.7, 72.0, 65.3, 64.8, 43.2, 37.1, 16.5.

HRMS (ES+TOF) calcd for [C₂₂ H₂₂O₉ S₄+H, M+H]⁺: 559.0225; found: 559.0241

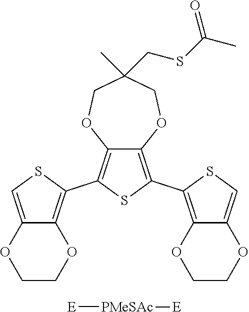

E-PMeOMs-E (2.8 g, 5.0 mmol, 1 eq) was dissolved in DMSO (50 mL) and Nal (1.1 g, 7.5 mmol, 1.5 eq) and KSAc (1.7 g, 15 mmol, 3 eq) was added. The solution was heated at 100° C. for 14 h, cooled and filtered through celite. Most of the DMF was removed under reduced pressure and the residue dissolved in EtOAc. The organics was subsequently washed with 1 M HCl and brine, dried over MgSO₄ and filtered. Silica was added and the solvent removed under vacuum. The product was purified through column chromatography (Pentane:DCM, Gradient from 5% DCM to 80%). Fractions containing product was evaporated, re-dissolved with minimal amount of DCM (5-10 mL) and slowly added into a stirred solution of pentane (200 mL). The pale-yellow precipitate was filtered and dried under vacuum. Yield: 85%.

¹H NMR (500 MHz, CDCl₃) δ 6.27; (2H, s), 4.35; (4H, m), 4.23; (4H, m), 4.06; (2H, d, J=12.1 Hz), 3.80; (2H, d,J=12.1 Hz), 3.25; (2H, s) 2.39; (3H, s), 0.99; (3H, s).

¹³C NMR (126 MHz, CDCl₃) δ 195.1, 144.5, 141.3, 137.4, 113.5, 110.4, 98.2, 78.3, 65.3, 64.8, 42.9, 33.3, 30.8, 18.2.

HRMS (ES+TOF) calcd for [C₂₃ H₂₂O₇S₄+H, M+H]⁺: 539.0321; found: 539.0201

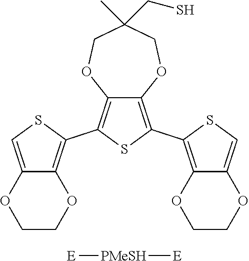

E-PMeOMs-E (810 mg, 1.5 mmol, 1 eq) was dissolved in dry DCM and cooled to −78° C. using an acetone/dry-ice bath. DIBAL-H (6 mL, 1M in hexanes, 4 eq) was slowly added and the solution stirred for 1 h at −78° C. The reaction was quenched by addition of 2M HCl (aq) and the mixture was allowed to warm to room temperature. Water was added and the organics separated, washed with water and brine. The organics was dried over MgSO4, filtered and mixed with silica. The solvent was removed under vacuum and the product was purified through column chromatography (Pentane:DCM, Gradient from 5% DCM to 80%). Fractions containing product was evaporated, re-dissolved with minimal amount of DCM and slowly added into a stirred solution of pentane. The pale-yellow precipitate was filtered and dried under vacuum. Yield: 90%.

¹H NMR (500 MHz, CDCl₃) δ 6.27; (2H, s), 4.35; (4H, m), 4.23; (4H, m), 4.20; (2H, d, J=12.1 Hz), 3.76; (2H, d, J=12.1 Hz), 2.90; (2H, d, J=9.0 Hz) 1.39; (1H, t,J=9.0 Hz), 0.96; (3H, s).

¹³C NMR (126 MHz, CDCl₃) δ 144.6, 141.4, 137.4, 113.6, 110.4, 98.2, 77.8, 65.3, 64.8, 42.9, 29.4, 17.7.

HRMS (ES+TOF) calcd for [C₂₁H₂₀O₆S₄+H, M+H]⁺: 497.0221; found: 497.0234

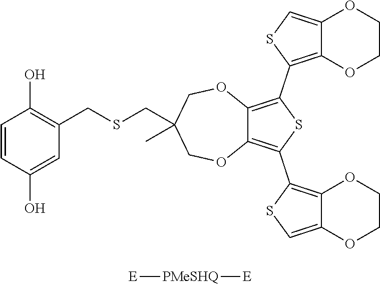

E-PMeSH-E (990 mg, 2 mmol, 1 eq) was dissolved in THF (50 mL) together with TIPS-protected bromomethyl-2,5-dihydroxybenzene (1.1 g, 2.2 mmol, 1.1 eq). TBAF (2.2 mL, 1 M in THF, 2.2 mmol, 2.2 eq) was added dropwise and the reaction stirred in r.t. over night. The volatiles was removed under vacuum and the residue diluted with DCM and washed with 1M HCl and brine. The organics was dried over MgSO4, filtered and mixed with silica. The solvent was removed under vacuum and the product was purified through column chromatography (Pentane:DCM, Gradient from 5% DCM to 80%). Fractions containing product was evaporated re-dissolved with minimal amount of DCM and slowly added into a stirred solution of pentane. The pale-yellow precipitate was filtered and dried under vacuum. Yield: 60%.

¹H NMR (500 MHz, CDCl₃) δ 6.75; (1H, d, J=8.4 Hz), 6.64; (2H, m) 6.27; (2H, s), 5.90; (2H, br), 4.34; (4H, m), 4.23; (4H, m), 4.17; (2H, d, J=11.9 Hz), 3.82; (2H, s), 3.71; (2H, d, J=11.9 Hz), 2.80; (2H, s), 0.96; (3H, s).

¹³C NMR (126 MHz, CDCl₃) δ 149.3, 149.1, 144.5, 141.4, 137.5, 124.0, 118.3, 117.7, 115.9, 113.7, 110.3, 98.3, 78.1, 65.3, 64.8, 43.4, 35.7, 34.3, 18.6.

HRMS (ES+TOF) calcd for [C₂₈H₂₆O₈ S₄+H, M+H]⁺: 619.0589; found: 619.0596

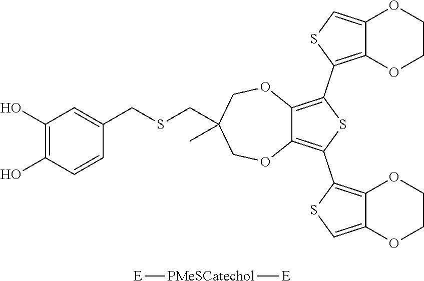

E-PMeSH-E (990 mg, 2 mmol, 1 eq) was disolved in THF (50 mL) together with 3,4-bis((tert-butyldimethylsilyl)oxy)benzyl bromide (1.1 g, 2.2 mmol, 1.1 eq, prepared as reported in J. Org. Chem. 1995, 60, 5, 1233). TBAF (2.2 mL, 1 M in THF, 2.2 mmol, 2.2 eq) was added dropwise and the reaction stirred in r.t. over night. The volatiles were removed under vacuum and the residue diluted with DCM and washed with 1M HCl and brine. The organics was dried over MgSO4, filtered and mixed with silica. The solvent was removed under vacuum and the product was purified through column chromatography (Pentane:DCM, Gradient from 5% DCM to 80%). Fractions containing product was evaporated re-dissolved with minimal amount of DCM and slowly added into a stirred solution of pentane. The pale-yellow precipitate was filtered and dried under vacuum. Yield: 60%.

¹H NMR (500 MHz, CDCl₃) δ 6.90; (1H, s), 6.77; (2H, m), 6.27; (2H, s), 5.10; (1H, s), 5.08; (1H, s), 4.35; (4H, m), 4.24; (4H, m), 4.16; (2H, d,J=11.8 Hz), 3.72; (d, J=11.8 Hz), 3.71; (2H, s), 2.78; (2H, s), 0.96; (3H, s).

¹³C NMR (126 MHz, CDCl₃) δ 144.7, 143.4, 142.8, 141.4, 137.4, 131.5, 122.0, 116.2, 115.5, 113.5, 110.4, 98.2, 78.3, 65.3, 64.8, 43.3, 37.6, 36.6, 18.8.

HRMS (ES+TOF) calcd for [C₂₈H₂₅O₈S₄+H, M+H]⁺: 619.0589; found: 619.0570

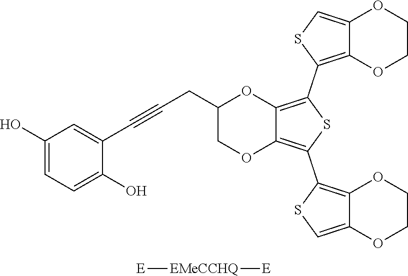

THP protected pyEDOT derivate (2.92 g, 6.4 mmol, 1 eq) was dissolved in DCM (100 ml). The solution was degassed by purging with argon for several minutes. N -Bromosuccinimide (2.41 g, 13.4 mmol, 2.1 eq) was added, and the solution was stirred for 25 min in r.t. The solvent was removed under reduced pressure. Column chromatography (Pentane:EtOAc 0% to 5% EtOAc) afforded a white powder (76%) which was used directly in the following step.

¹H-NMR (500 MHz, CDCl₃) δ 7.07; (1H, d, J=2.9 Hz, 1 H), 7.03; (1H, d, J=9.0 Hz,1 H), 6.95; (1H, dd, J=9.0, 2.9 Hz,), 5.37; (1H, m), 5.30; (1H, m), 7.03; (1H, dt, J=11.6, 2.1 Hz,), 4.45-4.36; (1H, m), 4.28-4.19; (1H, m), 3.92; (2H, m), 3.64-3.54; (2H, m), 3.01; (1H, ddd, J=17.0, 4.7, 1.2 Hz), 2.81; (1H, ddd, J=17.0, 8.8, 1.0 Hz), 2.03-1.92; (2H, m), 1.91-1.78; (4H, m), 1.74-1.53; (6H, m).

The THP protected dibromo pyEDOT (3.1 g, 5 mmol, 1 eq) and 2-(tributylstannyl)ethylenedioxythiophene (4.7 g, 11 mmol, 2.2 eq) was placed in round bottle flask containing anhydrous DMF (50 mL) and degassed by bubbling Ar through the solution for 20 min. Then, Pd(PPh₃)₄ (867 mg, 0.75 mmol, 7.5 mol %) was added and the reaction flask placed in a pre-heated metal block (120° C.). The solution was stirred under Ar for 16 h. The DMF was removed under reduced pressure and the residue dissolved in EtOAc (150 mL) and filtered through celite. The organics was extracted with 1M HCl (aq), brine and the organics dried over MgSO4. Silica was added and the solvent removed under vacuum. The residue was crudely purified through column chromatography (Pentane:EtOAc, Gradient from 5% EtOAc to 80%). Fractions containing product was dissolved in a DCM/MeOH mixture (3:1, 50 mL) and pyridinium p-toluensulfonate (30 mg) was added. The solution was stirred for 2 h in r.t., then silica was added and the solvent was removed under vacuum. The product was purified through column chromatography (Pentane:DCM, Gradient from 5% DCM to 80%). Fractions containing product was evaporated, re-dissolved with minimal amount of DCM (10 mL) and slowly added into a stirred solution of pentane (150 mL). The pale-yellow precipitate was filtered and dried under vacuum. Yield: 40%.

¹H NMR (500 MHz, CDCl₃) δ 6.79; (2H, m), 6.74; (1H, dd, J=8.7, 2.9 Hz), 6.28; (1H, s), 6.27; (1H,s), 5.43; (1H, s), 4.65; (1H, s), 4.57; (1H, d, J=6.6 Hz), 4.49; (1H, dd, J=11.5, 1.9 Hz), 4.35; (4H, m), 4.24; (5H, m), 3.04; (1H m), 2.97; (1H, m).

¹³C NMR (126 MHz, CDCl₃) δ 151.3, 148.8, 141.4, 137.1, 136.0, 118.0, 117.8, 115.7, 110.2, 110.1, 109.8, 108.6, 108.4, 98.0, 98.0, 91.3, 77.7, 72.4, 67.7, 65.5, 64.8, 22.5.

HRMS (ES+TOF) calcd for [C₂₇H₂₀O₈S₃+H, M+H]⁺: 569.0399; found: 569.0402.

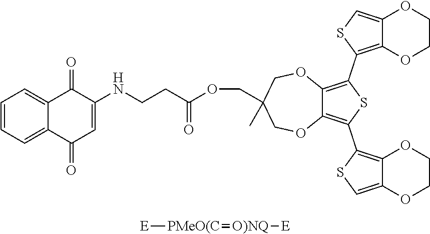

E-PMeOH-E (360 mg, 0.75 mmol, 1 eq) was dissolved in THF (25 mL) together with 4-3N-(1,4-dioxo-1,4-dihydronaphthalen-2-yl)propanoic acid (490 mg, 2.0 mmol, 2.7 eq, prepared as in Molecules 2014, 19, 9, 13188), DMAP (244 mg, 2.0 mmol, 2.7 eq) and DCC (412 mg, 2.0 mmol, 2.7 eq). The solution was stired in r.t. over night. and then filtered. Silica was added and the solvent removed under vacuum. The residue was purified through column chromatography (Pentane:EtOAc, Gradient from 5% EtOAc to 60%). Fractions containing product was evaporated, re-dissolved with minimal amount of DCM (10 mL) and slowly added into a stirred solution of pentane (150 mL). The pale-yellow precipitate was filtered and dried under vacuum. Yield 65% as a yellow powder.

¹H NMR (500 MHz, CDCl₃) δ 8.10; (1H, d, J=7.8 Hz), 8.03; (1H, d, J=7.8 Hz,), 7.72; (1H, t, J=7.5

Hz), 7.61; (1H, t, J=7.5 Hz), 6.24; (2H, s), 6.18; (1H, br s), 5.75; (1H, s), 4.36; (2H, s), 4.34; (4H, m), 4.28-4.16; (6H, m), 3.81; (2H, d, J=12.1 Hz), 3.54; (m, 2H), 2.76; (2H, t, J=6.3 Hz), 1.00; (3H, s).

¹³C NMR (126 MHz, CDCl₃) δ 183.2, 181.7, 171.2, 147.7, 144.3, 141.4, 137.5, 134.9, 133.6, 132.2, 132.1, 130.7, 126.5, 126.3, 113.5, 110.3, 101.5, 98.3, 76.5, 67.3, 65.2, 64.7, 42.8, 39.0, 34.1, 17.2.

HRMS (ES+TOF) calcd for [C₃₄H₂₉NO₁₀S₃+H, M+H]⁺: 708.1026; found: 708.1053.

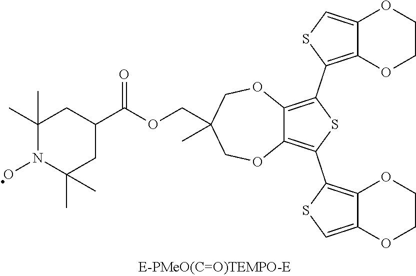 [SMILES: CC1(COC(=O)C2CC(C)(C)N(O)C(C)(C)C2)COc2c(-c3scc4c3OCCO4)sc(-c3scc4c3OCCO4)c2OC1]

E-PMeOH-E (360 mg, 0.75 mmol, 1 eq) was dissolved in THF (25 mL) together with 4-carboxy-TEMPO, free radical (400 mg, 2.0 mmol, 2.7 eq), DMAP (244 mg, 2.0 mmol, 2.7 eq) and DCC (412 mg, 2.0 mmol, 2.7 eq). The solution was stired in r.t. over night. and then filtered. Silica was added and the solvent removed under vacuum. The residue was purified through column chromatography (Pentane:EtOAc, Gradient from 5% EtOAc to 60%). Fractions containing product was evaporated, re-dissolved with minimal amount of DCM (10 mL) and slowly added into a stirred solution of pentane (150 mL). The pale-yellow precipitate was filtered and dried under vacuum. Yield 65% as a yellow powder.

Due to the presence of the free radical no NMR data could be obtained.

HRMS (ES+TOF) calcd for [C₃₁H₃₆NO₉S₃+H, M+H]⁺: 663.1630; found: 663.1650

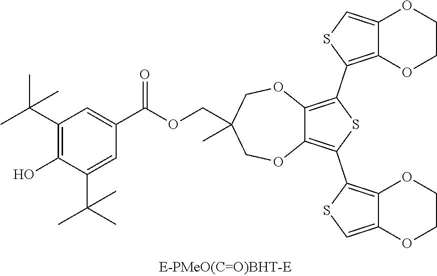 [SMILES: CC1(COC(=O)c2cc(C(C)(C)C)c(O)c(C(C)(C)C)c2)COc2c(-c3scc4c3OCCO4)sc(-c3scc4c3OCCO4)c2OC1]

E-PMeOH-E (360 mg, 0.75 mmol, 1 eq) was dissolved in THF (25 mL) together with 3,5-di-tert-butyl-4-hydroxybenzoic acid (500 mg, 2.0 mmol, 2.7 eq), DMAP (244 mg, 2.0 mmol, 2.7 eq) and DCC (412 mg, 2.0 mmol, 2.7 eq). The solution was stired in r.t. over night. and then filtered. Silica was added and the solvent removed under vacuum. The residue was purified through column chromatography (Pentane:EtOAc, Gradient from 5% EtOAc to 60%). Fractions containing product was evaporated, re-dissolved with minimal amount of DCM (10 mL) and slowly added into a stirred solution of pentane (150 mL). The pale-yellow precipitate was filtered and dried under vacuum. Yield 50% as a beige powder.

¹H NMR (500 MHz, CDCl₃) δ 7.93; (2H, s), 6.26; (2H, d, J=2.7 Hz), 5.68; (1H, s), 4.48; (2H, s), 4.35; (4H, m), 4.27; (2H, d, J=12.1 Hz), 4.24; (4H, m), 3.91; (2H, d, J=12.1 Hz), 1.46; (18H, s), 1.12; (3H, s).

¹³C NMR (126 MHz, CDCl₃) 6 167.0, 158.5, 144.5, 141.3, 137.4, 135.9, 127.3, 121.1, 113.3, 110.4, 98.2, 76.8, 66.5, 65.3, 64.8, 43.3, 34.5, 34.1, 30.1, 25.7, 25.1, 17.4.

HRMS (ES+TOF) calcd for [C₃₆H₄₀O₉S₃+H, M+H]⁺: 713.1913; found: 713.1930

General procedure for the synthesis of substituted 1,4-Dihydroxyanthraquinones

To a 500 ml round bottle flask equipped with a magnet was added AlCl₃ (35 g) and NaCl (7 g). The flask was heated using a metal block at 190° C. until the solids melted. Then anhydride (25 mmol 1 eq) and hydroquinone (or 1,4-dimethoxybenzene) (25 mmol 1 eq) were rapidly added. The heating was continued for 15 min before the temperature of the metal block was lowered to 100° C. Crushed ice (300 mL) was slowly added in small portions until violent reaction subsided. Finally, water was added to the bright red solution together and thereafter 5 mL of conc HCl. The solution was heated for 2 h resulting in a bright red precipitate which was filtered off while still hot. The red powder was washed with water and dried under vacuum. The compounds were used in the subsequent steps without further purification.

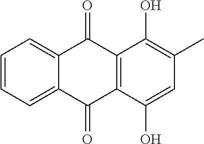 [SMILES: Cc1cc(O)c2c(c1O)C(=O)c1ccccc1C2=O]

1,4-dihydroxy-2-methylanthraquinone

Red solid, yield 62%

¹H NMR (500 MHz, d₆-DMSO) δ 13.19 (1H, s), 12.79; (1H, s), 8.25; (2H, m), 7.95; (2H, m), 7.35; (1H, m), 2.35; (3H, d, J=0.9 Hz).

Consistent with Chem Nat Compd 2017, 53, 949

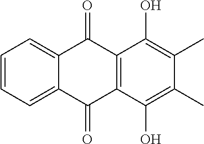 [SMILES: Cc1c(C)c(O)c2c(c1O)C(=O)c1ccccc1C2=O]

1,4-Dihydroxy-2,3-dimethylanthraquinone

Red solid, yield 65%

¹H NMR (500 MHz, d₆-DMSO) δ 13.50; (2H, s), 8.25; (2H, m), 7.95; (2H, m), 2.26; (6H, s).

Consistent with J. Am. Chem. Soc. 1981, 103, 8, 1992

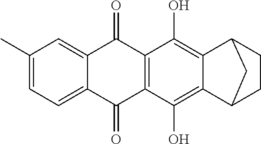 [SMILES: Cc1ccc2c(c1)C(=O)c1c(O)c3c(c(O)c1C2=O)C1CCC3C1]

5,12-dihydroxy-8-methyl-1,2,3,4-tetrahydro-1,4-methanotetracene-6,11-dione

Red solid, yield 60%

¹H NMR (500 MHz, CDCl₃) δ 13.14; (1H, s), 13.11; (1H, s), 8.21; (1H, d, J=7.9 Hz), 8.11; (1H, s), 7.59; (1H, d, J=7.9 Hz), 3.81; (2H, s), 2.54; (3H, s), 2.02; (2H, m), 1.81; (1H, d, J=9.0 Hz), 1.60; (1H, d, J=9.0 Hz), 1.27; (2H, m).

HRMS (ES+TOF) calcd for [C₂₀H₁₆O₄+H, M+H]⁺: 321.1127; found: 321.1119

### General Procedure for the Acetyl Protection

The quinone was dissolved in a mixture of acetic anhydride (75 ml) and pyridine (25 ml) and heated at 70° C. for 14 h. The solvent was removed under vacuum and toluene was added to remove traces of pyridine through co-evaporation. This was repeated until the pyridine smell disappeared. The dark solid was pure on NMR, however further purification through crystallization produced a pale-yellow powder.

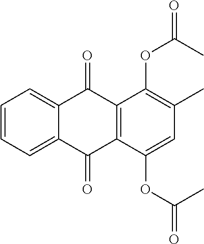 [SMILES: CC(=O)Oc1cc(C)c(OC(C)=O)c2c1C(=O)c1ccccc1C2=O]

1,4-diacetoxy-2-methylanthraquinone

Pale-yellow powder, recrystallized from CC14. Yield: 79%.

¹H NMR (500 MHz, CDCl₃) δ 8.16; (2H, m), 7.74; (2H, m), 7.31; (1H, s), 2.52; (3H, s), 2.49; (3H, s), 2.33; (3H, s).

¹³C NMR (126 MHz, CDCl₃) δ 182.0, 181.6, 169.7, 169.2, 147.8, 147.0, 141.3, 134.1, 134.1, 133.6, 133.5, 132.1, 127.0, 126.9, 21.3, 21.1, 16.9.

HRMS (ES+TOF) calcd for [C₁₉H₁₄O₆+H, M+H]⁺: 339.0874; found: 339.0869.

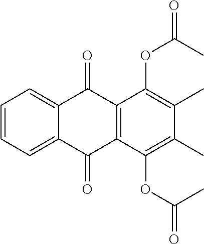 [SMILES: CC(=O)Oc1c(C)c(C)c(OC(C)=O)c2c1C(=O)c1ccccc1C2=O]

1,4-diacetoxy-2,3-dimethylanthraquinone

Pale-yellow powder, recrystalized from acetone. Yield: 80%.

¹H NMR (500 MHz, CDCl₃) δ 8.15; (2H, m), 7.72; (2H, m), 2.53; (6H, s), 2.27; (6H, s).

¹³C NMR (126 MHz, CDCl₃) δ 182.0, 169.5, 146.4, 140.0, 134.0, 133.7, 126.9, 123.5, 21.2, 13.8.

HRMS (ES+TOF) calcd for [C₂₀H₁₆O₆+H, M+H]⁺: 353.1021; found: 353.1025.

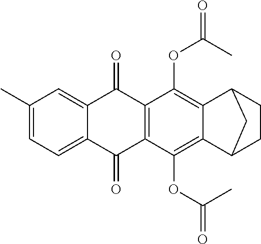 [SMILES: CC(=O)Oc1c2c(c(OC(C)=O)c3c1C1CCC3C1)C(=O)c1cc(C)ccc1C2=O]

8-methyl-6,11-dioxo-1,2,3,4,6,11-hexahydro-1,4-methanotetracene-5,12-diyl diacetate

Pale-yellow powder, recrystalized from acetone. Yield: 85%.

¹H NMR (500 MHz, CDCl₃) δ 8.03; (1H, d, J=7.9 Hz), 7.93; (1H, s), 7.51; (1H, d, J=7.9 Hz), 3.60; (2H, s), 2.51; (6H, s), 2.48; (3H, s), 1.98; (2H, m), 1.88; (1H, d, J=9.4 Hz), 1.62; (1H, d, J=9.4 Hz), 1.32; (2H, m).

¹³C NMR (126 MHz, CDCl₃) δ 182.8, 182.3, 169.6, 169.6, 149.9, 149.7, 145.0, 141.6, 141.5, 134.8, 133.5, 131.4, 127.0, 125.1, 125.1, 48.7, 41.4, 41.4, 25.7, 21.9, 21.2.

HRMS (ES+TOF) calcd for [C₂₄H₂OO₆+H, M+H]⁺: 405.1338; found: 421.1340

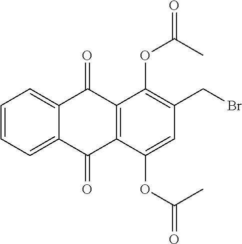 [SMILES: CC(=O)Oc1cc(CBr)c(OC(C)=O)c2c1C(=O)c1ccccc1C2=O]

8-(bromomethyl)-6,11-dioxo-1,2,3,4,6,11-hexa hydro-1,4-methanotetracene-5,12-diyl diacetate

1,4-diacetoxy-2-methylanthraquinone (3.39 g, 10 mmol,1 eq) was dissolved in dry CCl₄ (75 mL) and heated to 70° C. under Ar, NBS (1.78 g, 11 mmol, 1.1 eq) and AIBN (164 mg, 1 mmol,0.1 eq) was added in one portion and the reaction heated for 16 h. The reaction was cooled to room temperature and the solid filtered and washed with CCl₄. This solid contained succinimide and mostly product. Dissolving the solid in EtOAc and washing repeatedly with a solution of NaHCO₃ (sat) and brine gave, after drying over MgSO₄ and removal of solvent, pure product. The filtrate contained a mixture of unreacted starting material and product which could be enriched through repeated crystallization from CC14. Yield 60%.

¹H NMR (500 MHz, CDCl₃) δ 8.16; (2H, m), 7.74; (2H, m), 7.52; (1H, s), 4.44; (2H, br s), 2.55; (3H, s), 2.49; (3H, s).

HRMS (ES+TOF) calcd for [C₁₉H₁₃O₆Br+H, M+H]⁺: 416.9974; found: 416.9980.

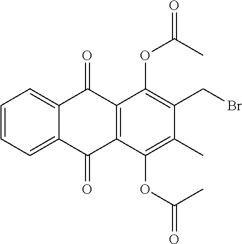 [SMILES: CC(=O)Oc1c(C)c(CBr)c(OC(C)=O)c2c1C(=O)c1ccccc1C2=O]

2-bromomethyl-1,4-diacetoxy-3-methylanthraquinone

1,4-Diacetoxy-2,3-dimethylanthraquinone (2.70 g, 7.7 mmol, 1 eq) was dissolved in dry CCl₄ (75 mL) and heated to 70° C. under Ar, NBS (1.50 g, 8.4 mmol, 1.1 eq) and AIBN (185 mg, 0.77 mmol, 0.1 eq) was added in one portion and the reaction stirred for 16 h. The reaction was cooled to room temperature and the solid filtered and washed with CC1₄. The solid contained succinimide as well as product and 2,3-dibromomethyl-1,4-diacetoxyanthraquinone while the filtrate contained a mixture of starting material and product. Dissolving the solid in EtOAc and washing repeatedly with a solution of NaHCO3 (sat) removed the succinimide and repeated crystallization from CCl₄ gave 2-bromomethyl-1,4-diacetoxy-3-methylanthraquinone containing 10% of dibromo derivative. Yield 40%.

¹H NMR (500 MHz, CDCl₃) δ 8.15; (2H, m), 7.72; (2H, m), 4.50; (2H, br s) 2.57; (3H, s), 2.53; (3H, s), 2.39; (3H, s).

HRMS (ES+TOF) calcd for [C₂₀H₁₆O₆Br+H, M+H]⁺: 431.0130; found: 431.0144.

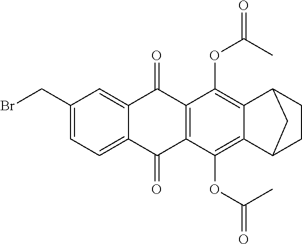 [SMILES: CC(=O)Oc1c2c(c(OC(C)=O)c3c1C1CCC3C1)C(=O)c1cc(CBr)ccc1C2=O]

8-(bromomethyl)-6,11-dioxo-1,2,3,4,6,11-hexahydro-1,4-methanotetracene-5,12-diyldiacetate 8-methyl-6,11-dioxo-1,2,3,4,6,11-hexahydro-1,4-methanotetracene-5,12-diyldiacetate (2.56 g, 6.3 mmol, 1 eq) was added to dry tetrachloroethylene (100 mL) and heated to 80° C. NBS (1.24 g, 7.0 mmol, 1.1 eq) and AIBN (103 mg, 0.63 mmol, 0.1 eq) was added in one portion and the solution heated at 80° C. for 16 h. The solution was cooled and the filtered to remove succinimide. The organics was diluted with DCM and washed sequentially with NaHCO₃ (sat) and dried over MgSO₄. After removal of solvent under reduced pressure a mixture of starting material, mono-brominated product and a small amount of 9,9-dibromomethyl-2,3-Norbornane-1,4-diacetoxyanthraquinone remained. The mono-brominated product proved difficult to separate and the mixture was used in the subsequent step without further purification.

¹H NMR (500 MHz, CDC1₃) 6 8.14 (1H, s), 8.12 (1H, d, J =7.5 Hz), 7.73 (1H, d, J =7.5 Hz) 4.53 (2H, s), 3.60 (2H, s), 2.51 (6H, s), 1.99 (2H, m), 1.88 (1H, d, J =9.4 Hz), 1.62 (1H, d, J =9.4 Hz), 1.32 (2H, m).

Note: The dibromo compound has a characteristic proton at 6.69 (1H, s) ppm.

HRMS (ES+TOF) calcd for [C₂₄ H₁₉O₆Br+H, M +H]⁺: 483.0443; found: 483.0464.

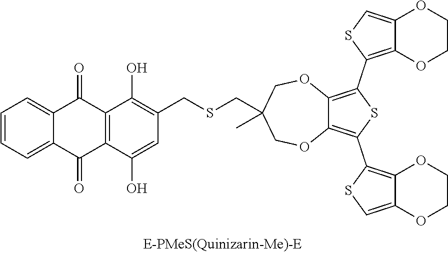 [SMILES: CC1(CSCc2cc(O)c3c(c2O)C(=O)c2ccccc2C3=O)COc2c(-c3scc4c3OCCO4)sc(-c3scc4c3OCCO4)c2OC1]

E-PMeSH-E (260 mg, 0.52 mmol, 1 eq) and 2-bromomethyl-1,4-diacetoxyanthraquinone (225 mg, 0.52 mmol, 1 eq) was mixed in degassed and anhydrous DCM (20 mL). DBU (0.11 mL, 0.75 mmol, 1.5 eq) was added and the solution heated at 40° C. for 3 h. Silica was added to the reaction mixture and the solvent was removed under vacuum and the product was purified through column chromatography (Pentane:EtOAc, Gradient from 10% EtoAc in pentane to 100% EtOAc) This yielded a beige solid.

The acetyl groups were removed by adding diethylamine (10 eq) to the solid (1 eq) dissolved in DCM (20 ml) and heating at 40° C. for 2 h. Subsequently, silica was added to the mixture and the volatiles removed under vacuum. Column chromatography (Pentane:DCM, Gradient from 30% DCM to 100%) gave a red solid. The solid was dissolved in minimal amount of DCM (5 mL) and added dropwise to a stirred solution of pentane (150 mL) to precipitate a red powder which was subsequently filtered off and dried. Yield 30%.

¹H NMR (500 MHz, CDCl₃) δ 13.45; (1H, s), 12.83; (1H, s), 8.31; (2H, m) , 7.80; (2H, m), 7.31; (1H, s), 6.17; (2H, s), 4.33; (4H, m), 4.22; (4H, m), 4.20; (2H, d, J=11.8 Hz), 3.90; (2H, s), 3.67; (2H, d, J=11.8 Hz), 3.01; (2H, s), 0.95; (3H, s).

¹³C NMR (126 MHz, CDCl₃) δ 187.2, 186.4, 157.6, 156.6, 144.5, 141.3, 141.2, 140.5, 137.3, 134.4, 134.3, 133.7, 133.6, 128.6, 127.1, 126.9, 113.7, 112.8, 112.1, 110.4, 98.2, 98.2, 78.1, 65.2, 64.7, 46.3, 43.7, 43.3, 37.5, 32.0, 18.5.

HRMS (ES+TOF) calcd for [C₃₆H₂₈O₁₀S₄+H, M+H]⁺: 749.0644; found: 749.0650.

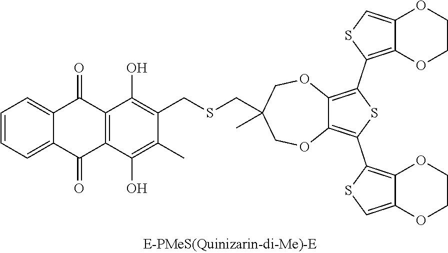 [SMILES: Cc1c(O)c2c(c(O)c1CSCC1(C)COc3c(-c4scc5c4OCCO5)sc(-c4scc5c4OCCO5)c3OC1)C(=O)c1ccccc1C2=O]

E-PMeSH-E (409 mg, 0.83 mmol, 1.1 eq) and 2-bromomethyl-3-methyl-1,4-diacetoxyanthraquinone (323 mg, 0.75 mmol, 1 eq) was mixed in degassed and anhydrous DCM (20 mL). DBU (0.17 mL, 1.1 mmol, 1.5 eq) was added and the solution heated at 40° C. for 3 h. Silica was added to the reaction mixture and the solvent was removed under vacuum and the product was purified through column chromatography (Pentane:EtOAc, Gradient from 10% EtoAc in pentane to 100% EtOAc) This yielded a beige solid

The acetyl groups were removed by adding diethylamine (10 eq) to the solid (1 eq) dissolved in DCM (20 ml) and heating at 40° C. for 2 h. Subsequently, silica was added to the mixture and the volatiles removed under vacuum. Column chromatography (Pentane:DCM, Gradient from 30% DCM to 100%) gave a red solid. The solid was dissolved in minimal amount of DCM (5 mL) and added dropwise to a stirred solution of pentane (150 mL) to precipitate a red powder which was subsequently filtered off and dried. Yield 20%.

¹H NMR (500 MHz, CDCl₃) δ 13.70; (1H, s), 13.50; (1H, s), 8.32; (2H, m) , 7.80; (2H, m), 6.21; (2H, s), 4.33; (4H, m), 4.22; (4H, m), 4.20; (2H, d, J=12.3 Hz), 4.05; (2H, s), 3.70; (2H, d, J=12.3 Hz), 3.11; (2H, s), 2.46; (3H, s), 0.95; (3H, s).

¹³C NMR (126 MHz, CDCl₃) δ 186.8, 186.6, 157.3, 156.8, 144.6, 141.3, 139.4, 138.2, 137.3, 134.3, 134.2, 133.8, 127.0, 127.0, 113.7, 111.1, 110.7, 110.5, 98.2, 78.1, 65.2, 64.8, 43.5, 42.3, 38.1, 28.8, 18.4, 12.8, 11.4.

HRMS (ES+TOF) calcd for [C₃₇H30O₁₀S₄+H, M+H]⁺: 763.0800; found: 763.0800.

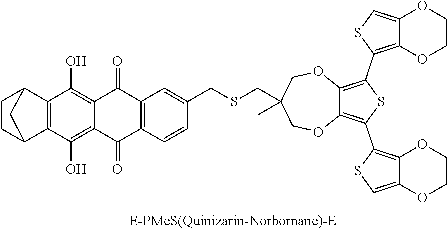 [SMILES: CC1(CSCc2ccc3c(c2)C(=O)c2c(O)c4c(c(O)c2C3=O)C2CCC4C2)COc2c(-c3scc4c3OCCO4)sc(-c3scc4c3OCCO4)c2OC1]

The mixture containing 8-(bromomethyl)-6,11-dioxo-1,2,3,4,6,11-hexahydro-1,4-methanotetracene-5,12-diyldiacetate (1 eq) was dissolved in DCM (25 mL) and degassed by bubbling Ar through the solution. DBU (1.2 eq) was added and the solution turned dark, thereafter E-PMeSH-E (1.1 eq) was added and the solution was heated at 40° C. for 2 h and in r.t. for a further 14 h. Silica was added to the mixture and the volatiles removed under vacuum. The product was separated from the non-brominated and di-brominated compounds through column chromatography (Pentane:EtOAc, Gradient from 5% EtOAc to 80%) to give a brown solid.

The acetyl groups were removed by adding diethylamine (10 eq) to the solid (1 eq) dissolved in DCM (20 ml) and heating at 40° C. for 2 h. Subsequently, silica was added to the mixture and the volatiles removed under vacuum. Column chromatography (Pentane:DCM, Gradient from 30% DCM to 100%) gave a red solid. The solid was dissolved in minimal amount of DCM (5 mL) and added dropwise to a stirred solution of pentane (150 mL) to precipitate a red powder which was subsequently filtered off and dried. Yield 25%.

¹H NMR (500 MHz, CDCl₃) δ 13.09; (1H, s), 13.06; (1H, s), 8.32; (1H, s), 8.19; (1H, d, J=8.0 Hz), 7.73; (1H, d, J=8.0 Hz), 6.20; (2H, s), 4.34; (4H, m), 4.23; (4H, m), 4.15; (2H, d, J=11.2 Hz), 3.94; (2H, s), 3.80; (2H, s), 3.62; (2H, d, J=11.2 Hz), 2.90; (2H, s), 2.01; (2H, m), 1.81; (1H, d,J=9.4 Hz), 1.61; (1H, d, J=9.4 Hz), 1.30; (2H, m), 0.91; (3H, s).

¹³C NMR (126 MHz, CDCl₃) δ 187.1, 186.8, 152.9, 152.8, 148.1, 147.9, 145.8, 144.4, 141.3, 137.4, 134.7, 134.0, 132.5, 127.5, 127.5, 113.8, 112.6, 112.4, 110.3, 110.3, 98.2, 98.2, 78.0, 65.2, 64.7, 49.2, 43.2, 40.8, 40.8, 37.7, 36.5, 25.7, 18.5.

HRMS (ES+TOF) calcd for [C₄₁H₃₄O₁₀S₄+H, M+H]⁺:815.1113; found: 815.1120.

### Experimental Data for poly-E-PMeSHQ-E

### Chemical Polymerisation of E-PMeSHQ-E

10 mg of E-PMeSHQ-E was dissolved in 500 μl acetonitrile and separately 2,2,6,6-tetramethyl-1-oxopiperidinium tosylate (2 eq, 12 mg) was dissolved in 500 μl dichloromethane. The oligomer solution and oxidant solution were mixed, yielding a stable suspension of poly-E-PMeSHQ-E. Note that oxidation of a corresponding monomer solution with such mild oxidants is generally not possible or proceeds impractically slow.

### Electrochemical Studies of poly-E-PMeSHQ-E

The polymer suspension obtained (10 μl corresponding to 0.1 mg of oligomer) was drop-cast on an electrode (glassy carbon, graphite, or gold interdigitated array electrode). The resulting polymer film was allowed to dry at room temperature under vacuum.

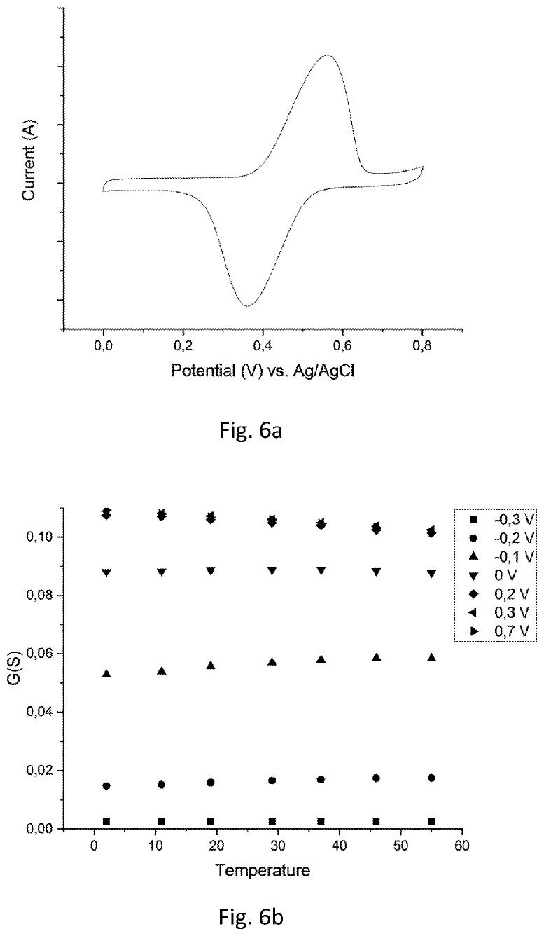

The electrochemical properties of the film have been investigated in 0.5M sulphuric acid solution. FIG. 6a shows a cyclic voltammogram of a glassy carbon electrode coated with the poly-E-PMeSHQ-E as described above. It can been seen that the redox peaks that are obtained corresponds to the redox reaction of the pendant hydrooquinone group, and occur at the expected redox potential for hydroquinone. The capacity observed is close to 100% of the theoretical capacity.

Coating of an interdigitated array electrode as described above with the poly-E-PMeSHQ-E allows the electrical conductivity of the polymer to be measured as a function of both potential (redox state) and temperature. FIG. 6b shows the plot obtained from the interdigitated array electrode studies. It can be seen that at least for some redox states there is a negative dependence of conductivity on temperature. This indicates that the method of electron transport through the material is non-thermally activated.

### Electrochemical Post-Deposition Polymerisation of E-PMeSHQ-E

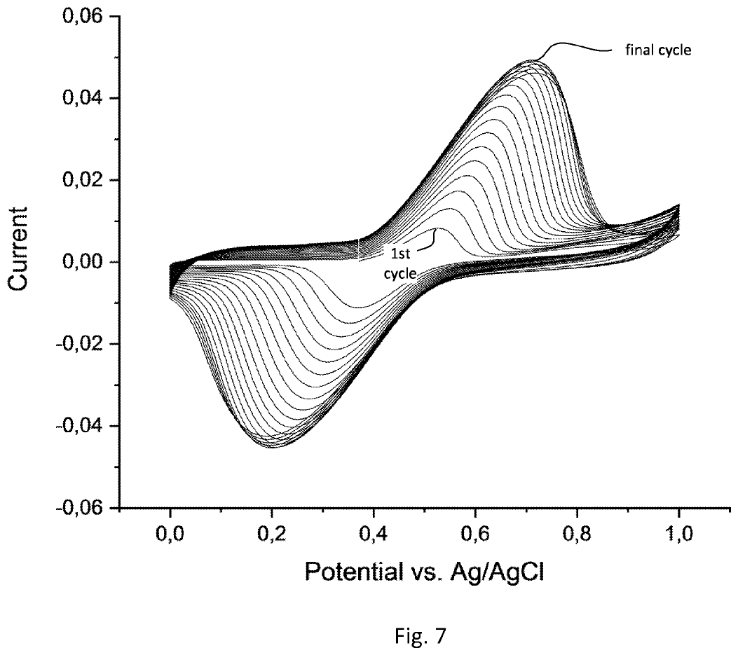

E-PMeSHQ-E (10 mg) was dissolved in 100 μl NMP and 10 μl of the resulting solution was drop-cast on a graphite substrate. Polymerisation is performed electrochemically by cyclic voltammetry of the substrate to successively higher upper potentials up to a potential of 1V vs Ag/AgCl. FIG. 7 shows the obtained cyclic voltammogram. A clear increase in capacity from the quinone can be seen as the number of cycles performed progresses. This indicates that the composition becomes more and more conductive upon progression of the cyclic voltammetry due to oxidative polymerisation of the E-PMeSHQ-E oligomer to provide poly-E-PMeSHQ-E. The deposited film blackens as the cyclic voltammetry progresses, which is a further indication that polymerisation occurs.

### Experimental Data for poly-E-PMeO(C═O)NQ-E

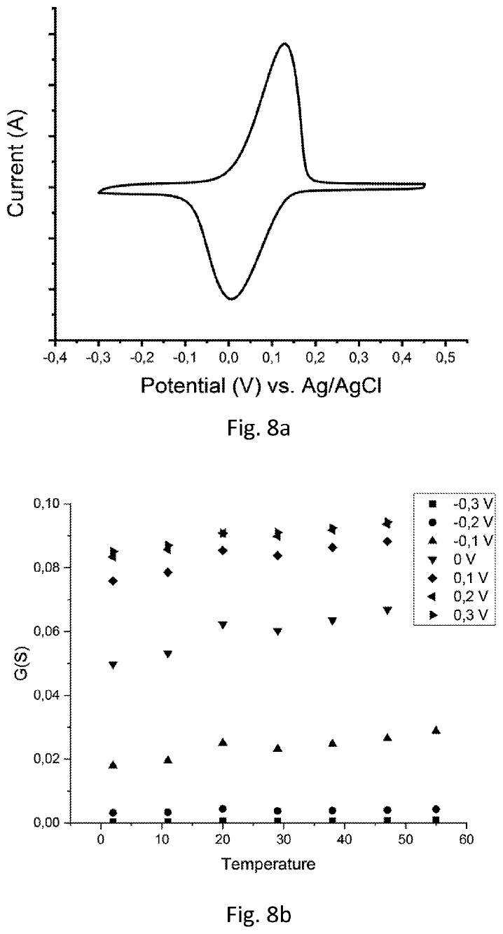

poly-E-PMeO(C═O)NQ-E was synthesised by 2,2,6,6-tetramethyl-1-oxopiperidinium tosylate oxidation as described above, and its electrochemical properties were investigated in a similar manner as to poly-E-PMeSHQ-E. FIG. 8a shows the cyclic voltammogram obtained from a glassy carbon electrode coated with poly-E-PMeO(C═O)NQ-E (performed in 0.5 M sulphuric acid), and FIG. 8b shows the results of the interdigitated array electrode experiments. The conclusions reached from these experiments correspond well to the conclusions drawn as for poly-E-PMeSHQ-E (mutatis mutandis).

### Experimental Data for E-EMeCCHQ-E

E-EMeCCHQ-E was applied to a gold disc electrode by drop casting from a 0.05 M MeCN solution of the trimer. The material was post-deposition-polymerized by cyclic voltammetry in 0.5 M H₂SO₄(aq). From the cyclic voltammetry, a successive build-up of electrochemical activity could be observed. Capacity build-up starts at 0.6 V vs Ag/AgCl. Initially capacity is built up over the quinone peak and down to −0.2 V. However as the polymerization proceeds the capacitance from the polymer extends down to, and below, −0.3 V indicating that the polymer doping is down-shifting as the conjugation length becomes larger, as expected during polymerization.

### Comparison of Monomer vs Oligomer

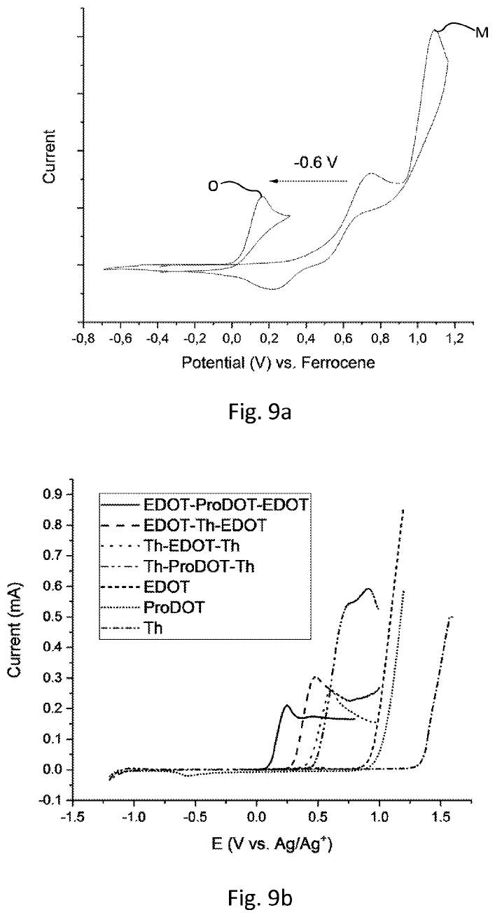

FIG. 9a shows a comparison of the polymerisation potential for monomeric EMeCCHQ (labelled M in the figure, synthesised as reported in Electrochimica Acta 2017 235, 356-364), as compared to the polymerisation potential of the oligomeric E-PMeSHQ-E (labelled O in the figure). Electro polymerisation is performed in acetonitrile solution with 0.1M TBAPF6 as supporting electrolyte. Oxidation for the trimer (E-PMeSHQ-E) occurs at 0 V vs. ferrocene as compared to 0.6 V for the monomer EMeCCHQ. Thus, it can be seen that oxidation of the oligomer occurs at significantly lower potentials than required for oxidation of the monomer. This corroborates that polymerisation of the oligomer can be performed under milder conditions than required for polymerisation of the monomer.

FIG. 9b shows the oxidation potential for a number of thiophene monomers and trimers. The trimers and monomers are listed in the key of the figure in order of increasing oxidation potential, with EDOT-ProDOT-EDOT having the lowest oxidation potential and Th having the highest oxidation potential. It can be seen that the monomers have the highest oxidation potential and are thus most difficult to polymerise. For the trimers, it can be seen that each additional alkylenedioxythiophene moiety decreases the oxidation potential, and trimers comprising at least two alkylenedioxythiophene moieties have low oxidation potential. This allows such trimers to be polymerised under mild conditions. For example, such trimers may be polymerised in aqueous electrolytes without risk for electrolyte degradation and formation of reactive oxygen species.

### Porous Composites

Porous materials can also be coated using the methods described herein. A solution of the relevant conducting redox oligomer (E-P(QH2)-E or E-P(NQ)-E) dissolved in NMP was prepared. A carbon felt substrate ((AvCarb G200 Soft Graphite Battery Felt, FuelCellStore) was dip-coated in the solution and dried under vacuum. Current conductors (Pt-wire) were attached to the coated substrate and the coated substrate was immersed in an electrolyte solution (0.5 M H₂SO₄). An oxidative potential was applied to the substrate, leading to polymerisation of the conducting redox oligomer, thus providing a porous felt substrate coated with the relevant conducting redox polymer. The coated sample was washed and dried prior to subsequent use.

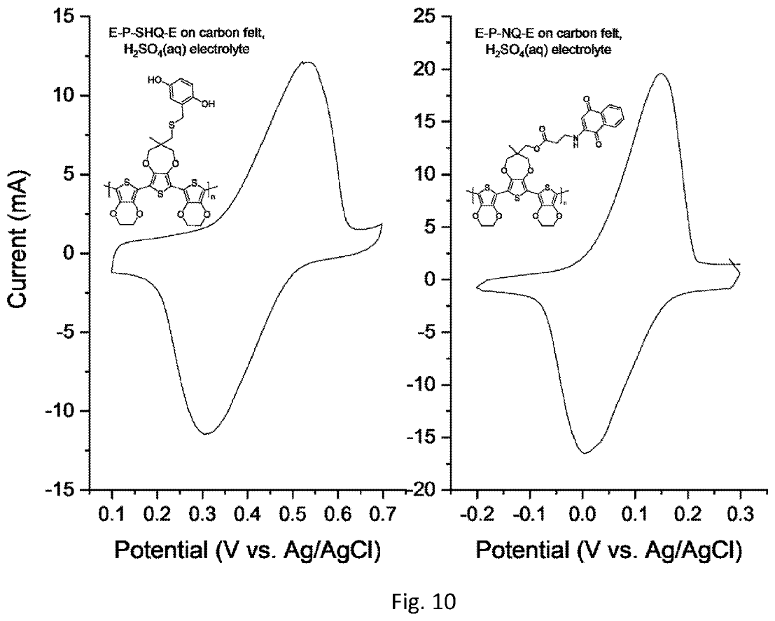

Cyclic voltammograms obtained from the carbon sponge substrate coated with the relevant conducting redox polymer are shown in FIG. 10.

### Organic Battery

An organic battery was constructed using the materials and methods as described herein. The cathode material consisted of pEP(QH2)E, which was formed by oxidative polymerization of EP(QH₂)E. Similarly, the anode material pEP(NQ)E was formed from EP(NQ)E. The electrode materials were directly deposited without conducting additives or binders. Conductivity was achieved from the poly-thiophene backbone being oxidized/doped, e.g. with HSO₄⁻. The battery was assembled as an all-organic proton battery using 0.5 M H₂SO₄ (aq) aqueous electrolyte, which enabled a rocking-chair motion of the protons. The anode and cathode redox activity relies on the two-electron two-proton (2e2H) redox process of the pendant groups Q and NQ. When the battery is charged, the pendant groups are in the Q and NQH₂ states, for the positive electrode (cathode) and negative electrode (anode), respectively. During discharge, the active cathode material is converted to QH₂ while the anode is converted to NQ.

E=3,4-ethylenedioxythiophene; NQ=naphthoquinone; NQH₂=naphthohydroquinone; P=3,4-propylenedioxythiophene; p=polymerized; Q=benzoquinone; QH₂=hydroquinone.

It was found that the battery characteristics were well captured by the combined properties of the two individual electrode materials. That is, the average cell voltage (0.4 V) corresponded to the difference in charge/discharge plateaus between pEP(QH₂)E and pEP(NQ)E and the capacity was comparable to the capacity of the limiting pEP(NQ)E electrode. The battery could be charged at constant current (galvanostatically) or at constant voltage (CV). When CV charging was used, corresponding to potential step charging in a three-electrode setup, at a voltage of 0.6 V for 100 s, the battery was charged as pEP(QH₂)E was oxidized to pEP(Q)E while pEP(NQ)E was reduced to pEP(NQH₂)E. The highest currents observed during the charging step were around 30 A/g, which was slightly lower than the currents for the individual electrodes; 100 s was required to fully charge the battery. This was attributed to the higher pressure inside the coin cell preventing swelling of the polymer upon charging. Nonetheless, the battery was charged to 50% within 10 s and 80% after 25 s. The resulting discharge capacity was around 60 mAh/g at 3 C, which is about 80% of the theoretical capacity of the pEP(NQ)E electrode (theoretical capacity 75 mAh/g). The battery retained 85% of its initial capacity after 500 cycles using constant voltage charging followed by galvanostatic discharge.

Due to the battery being able to be charged at constant voltage, it could be charged by direct integration with a commercial organic photovoltaic cell with a rated output of 0.6 V at 6-10 mA under full sun, without requiring additional electronics. The battery was fully charged in 100 s by simply connecting it to the solar cell exposed to a one sun equivalent light.

The possibility of using the battery at sub-zero temperatures for low temperature applications was also explored. In order to prevent freezing the electrolyte, the sulfuric acid concentration was increased from 0.5 M to 3.3 M thus inducing a freezing point depression to −27° C. With the higher electrolyte concentration galvanostatic cycling of the battery at −24° C. afforded a discharge capacity of 60 mAh/g up to 1.1 A/g and 40 mAh/g at 3 A/g. Hence, capacity and the rate capability were largely unaffected by the reduced temperature.

Finally, the batteries were used to power a thermometer chosen to demonstrate an application in, for example, monitoring packaging temperatures during transportation. Two batteries (containing ˜1 mg material/electrode) were coupled in series to achieve a higher voltage. The batteries powered the thermometer for more than one hour, with gradually fading display intensity.

1. A compound of formula IVa, or a salt thereof: 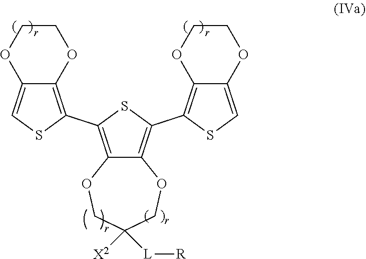
wherein
each instance of -L- is independently selected from a direct bond or a covalent linker moiety;
each instance of —R is independently a reversible redox group;
each instance of —X² is independently selected from -L-H, -L-T, or -L(-R)ₘ;
each instance of T is independently selected from —CN or —N₃;
each instance of m is independently selected from 1 to 5; and
each instance of r is independently selected from 0, 1 or 2.

2. Compound according to claim 1, wherein each instance of —X² is independently selected from —H, C₁-C₁₂ alkyl, or -L(-R)ₘ.

3. Compound according to claim 1, wherein each instance of —X² is independently selected from H or C₁-C₁₂ alkyl.

4. Compound according to claim 1, wherein each instance of -L- is independently selected from a covalent linker moiety having a structure —(CH₂)ₛ-G¹-(CH₂)ₜ-G²- or -G²-(CH₂)ₜ-G¹-(CH₂)ₛ— wherein s is from 0 to 6, t is from 0 to 6, and each of -G¹- and -G²- is independently selected from the group consisting of a direct bond, —O—, —S—, —SO₂—, —SO₃—, —O₃S—, —SO₂NH—, —NHSO₂—, —NH—, —N(C₁-C₆ alkyl)-, —C(O)—, —CO₂—, —O₂C—, —C(O)NH—, —NHC(O)—, —OC(o)O—, —NHC(O)NH—, —NHC(O)O—, —OC(O)NH——C≡C—, —CH═CH—, —Ph— and —Hy—.

5. Compound according to claim 1, wherein R is an organic redox group.

6. Compound according to claim 1, wherein R is selected from the group consisting of terephthalate, naphthoquinone, anthraquinone, catechol, quinone, quinizarin, naphthazarin, indigo, TEMPO, galvinoxyl, phenol, naphthalene diimide, pyrene diimide, perylene dimide, dibenzothiophenesulfone, or substituted derivatives thereof

7. Compound according to claim 1, wherein R is an organometallic redox catalyst.

8. A polymer comprising a repeating unit of formula RIVa, or a salt thereof: 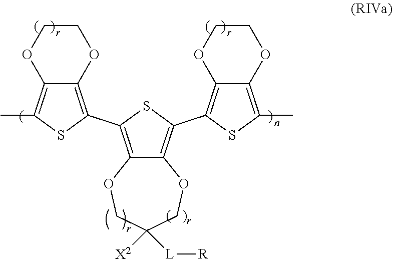
wherein:
n is from 2 to 5, such as 3 or 5, preferably 3;
each instance of —X² is independently selected from -L-H, -L-T, or -L(-R)ₘ;
each instance of -L- is independently selected from a direct bond or a covalent linker moiety;
each instance of -T is independently selected from —CN or —N₃;
each instance of -R is independently a reversible redox group;
each instance of r is independently selected from 0, 1 or 2; and
each instance of m is independently selected from 1 to 5.

9. Polymer according to claim 8, wherein each instance of X² is independently selected from H or C₁-C₁₂ alkyl.

10. A method of manufacturing a polymer-coated substrate, the method comprising the steps:
a) providing a substrate;
b) coating a compound according to claim 1 onto the substrate in order to produce a substrate having an oligomeric coating; and
c) polymerising the oligomeric coating by oxidative polymerization to provide a polymer-coated substrate.

11. A coating composition comprising a compound according to claim 1 dispersed in a carrier liquid.

12. A polymer-coated substrate comprising a polymer according to claim 8, wherein the substrate is a conducting current collector material.

13. Polymer-coated substrate according to claim 12, wherein the substrate is porous.

14. An organic battery comprising a compound according to claim 1, and/or a polymer comprising a repeating unit of formula RIVa, or a salt thereof: 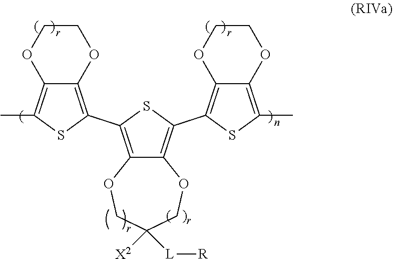
wherein:
n is from 2 to 5, such as 3 or 5, preferably 3;
each instance of —X² is independently selected from -L-H, -L-T, or -L(-R)ₘ;
each instance of -L- is independently selected from a direct bond or a covalent linker moiety;
each instance of -T is independently selected from —CN or —N₃;
each instance of -R is independently a reversible redox group;
each instance of r is independently selected from 0, 1 or 2; and
each instance of m is independently selected from 1 to 5;
and/or a polymer-coated substrate comprising a conducting current collector material.

15. An organic battery according to claim 14, further comprising an aqueous electrolyte.

16. The polymer-coated substrate of claim 12, wherein the conducting current collector material comprises graphite.
